SEQUENCE LISTING (2 sequences)

SEQ ID NO: 1 - DNA, 12 bp, synthetic construct
  source 1..12: mol_type = other DNA
gcgagcagttgt

SEQ ID NO: 2 - DNA, 12 bp, synthetic construct
  source 1..12: mol_type = other DNA
cgctcgtcddcd

(19) United States
(12) Patent Application Publication
(10) Pub. No.: US 2026/0131014 A1
(43) Pub. Date: May 14, 2026

(54) SUPRAMOLECULAR AGENTS DESIGN & USES THEREOF

(71) Applicants: UNIVERSITE DE GENEVE, GENEVA 4 (CH); THE UNIVERSITY OF SYDNEY, NEW SOUTH WALES (AU)

(72) Inventors: NICOLAS WINSSINGER, NYON (CH); MILLICENT DOCKERILL, VEIGY FONCENEX (FR); SIMONA ANGERANI, GENÈVE (CH); RICHARD PAYNE, ANNANDALE (AU)

(21) Appl. No.: 19/385,393

(22) Filed: Nov. 11, 2025

Related U.S. Application Data
(60) Provisional application No. 63/718,829, filed on Nov. 11, 2024.

(51) Int. Cl. A61K 47/64; A61K 47/54; A61P 7/02

(52) U.S. Cl. CPC A61K 47/64; A61K 47/549; A61P 7/02

(57) ABSTRACT

The present invention relates to supramolecular agents presenting on demand reversibility of activity and related methods. The invention in particular relates to supramolecular agents with anticoagulant activity which can be reversed with the use of an antidote agent.

### CROSS-REFERENCE TO RELATED APPLICATION

This application claims the benefit of U.S. Provisional Application Ser. No. 63/718,829, filed Nov. 11, 2024, the disclosure of which is hereby incorporated by reference in its entirety, including all figures, tables and amino acid or nucleic acid sequences.

### STATEMENT REGARDING SEQUENCE LISTING

The Sequence Listing for this application is labeled “Seq-List.xml” which was created on Oct. 24, 2025 and is 2,673 bytes. The entire content of the sequence listing is incorporated herein by reference in its entirety.

### FIELD OF THE INVENTION

The present invention relates to the design of supramolecular agents with on demand reversibility. The invention in particular relates to supramolecular agents with anticoagulant activity which can be reversed with the use of an antidote agent.

### BACKGROUND OF THE INVENTION

Drugs are administered at a dosing schedule set by their therapeutic index and termination of action is achieved by clearance and metabolism of the drug (hours to days for small molecules, weeks to months for biologics). In some cases, it is important to achieve a fast reversal of the drug's action. A case in point is for anticoagulant drugs.

For example, anticoagulants are critically important therapies for the prevention or reversal of thrombotic events in patients and achieve their effect by reducing fibrin deposition by inhibiting fibrinogen proteolysis and/or platelet activation (Di Nisio et al., 2005, N. Engl. J. Med. 353, 1028-1040; https://doi.org:DOI 10.1056/NEJMra044440). One of the key targets of anticoagulant therapy is the protease thrombin (Factor IIa/FIIa). The use of anticoagulants is based on a risks benefits analysis where prevention or reduction of progression of thromboembolic disease outweighs the increased risk of bleeding in adverse events or trauma. Indeed, many anticoagulants (particularly heparin and warfarin) (Hirsh, 2001, Circulation 103, 2994-3018; https://doi.org:Doi 10.1161/01.Cir.103.24.2994) require close clinical monitoring to prevent life threatening bleeding side effects. Despite this, it has been estimated that anticoagulant-related bleeding is responsible for 15% of all emergency hospital visits for adverse drug effects (Geller et al., 2020, J. Gen. Int. Med. 35, 371-373; https://doi.org:10.1007/s11606-019-05391-y). Life threatening bleeding is the most concerning complication of anticoagulant therapy and strategies for the reversal of anticoagulation are therefore essential (Thomas et al., 2018, Clin Med 18, 314-319; https://doi.org:DOI 10.7861/clinmedicine.18-4-314). A common strategy to reverse the effects of anticoagulants is the administration of non-specific reversal agents that involves the infusion of coagulation factors designed to overwhelm the effects of circulating anticoagulants (Smith et al., 2019, J Thromb Thrombolys 48, 250-255; https://doi.org:10.1007/s11239-019-01846-5; Ebright & Mousa, 2015, Clin. App. Thrombosis/Hemostasis, 21, 105-114; https://doi.org:10.1177/1076029614545211).

In clinical settings, unfractionated heparin is a useful anticoagulant since protamine sulfate can be used for rapid reversal, but unfractionated heparin requires close monitoring (Guerrini et al., 2008, Nat Biotechnol 26, 669-675; https://doi.org:10.1038/nbt1407; Eikelboom, et al., 2006, Thromb. Haemostasis 96, 547-552; https://doi.org:10.1160/Th06-05-0290).

More recently, monoclonal antibodies and recombinant FXa have been developed, which bind to a specific small molecule anticoagulant with high affinity (idarucizumab for dabigatran and andexanet alfa for apixaban, edoxaban, and rivaroxaban), thus reversing the inhibition of factor Xa (FXa) or thrombin (FIIa) (Pollack et al., 2015, N. Engl. J. Med. 373, 511-520; https://doi.org:10.1056/NEJMoa1502000; Lu et al., 2013, Nat. Med. 19, 446-451; https://doi.org/10.1038/nm.3102). Whilst these approaches are effective, there are limitations for their use and are associated with high cost.

Therefore, currently, there exists an evident need for synthetic molecules with on-demand switching of activity properties for use in prognostic and predictive medicine but also as therapeutics.

### SUMMARY OF THE INVENTION

The present invention is directed to the unexpected finding that it is possible to design the in vivo formation of supramolecular assemblies of active agents which are able to act cooperatively on a target once in the form of those supramolecular assemblies and achieve a certain biological effect on said target, while this biological effect can be reversed by the action of an antidote agent which would lead to the disassembly of the supramolecular assembly.

The design strategy is based on the ability to link active agents by hybridized peptide nucleic acid (PNA) strands—by a reversible supramolecular interaction—that are able to interact cooperatively with the target at two distinct sites (FIG. 1), with the formation of an active supramolecular assembly triggered by the target. Disruption of the supramolecular interaction between the active agents results in a loss of cooperativity between those, yielding a loss of biological activity.

This general strategy is illustrated herein by the design of supramolecular assemblies of thrombin-inhibiting anticoagulant agents. It is shown that the design of supramolecular assemblies of active agents that are able to interact cooperatively with the target at two distinct sites of thrombin (binary interactions directed to the active site of thrombin and to exosite II (the so-called heparin binding site)) leads to very potent bivalent direct thrombin inhibitors (Ki 74 pM) which exhibit both in vitro and in vivo potent anticoagulant activities. The formed direct bivalent thrombin inhibitor unexpectedly displayed an 800-fold gain in activity relative to individual active agents by taking advantage of the dynamic properties of supramolecular non-covalent association of the binary fragments through PNA hybridization. This activity can be reversed on demand by the use of an antidote agent (competing oligonucleotide or “antidote”) which leads to the disassembly of the supramolecular assembly.

The inventors have in particular designed a methodology allowing i) the formation of supramolecular assemblies of agents, once in presence of a target and wherein said agents cooperatively act on said target and ii) the disassembly of said supramolecular assemblies through the action of a dissociating agent leading to the loss of activity of the supramolecular assemblies, once disassembled.

An object of this invention is to provide an active therapeutic agent (e.g. a therapeutic inhibitor) which activity can be modulated by external factors, yielding a simple strategy for reversing its activity (e.g. inhibitory activity).

It is advantageous to provide a supramolecular assembly of agents wherein the supramolecular assembly results in a gain in activity relative to individual agents.

It is advantageous to provide an active supramolecular assembly of agents which activity can be reversed on demand by external factors in a controlled manner, i.e. allowing simple tuning of the dissociation kinetics of the components of the supramolecular assembly, in particular when the activity of the supramolecular assembly may lead to side effects.

It is an object of the invention to provide new method of preventing or treating disorders by a supramolecular assembly formed in presence of a target and which activity can be reversed through the action of an antidote agent which leads to the disassembly of the supramolecular assembly.

Objects of this invention have been achieved by providing methods, formulations, supramolecular assemblies and uses thereof according to the invention.

Disclosed herein, according to a first aspect of the invention, is a method of preventing or treating a disorder in a subject in need thereof, said method comprising the steps of:
Providing a first targeting agent wherein said first targeting agent comprises i) a first binding moiety that has a binding affinity to a first site of a biological target and ii) a first PNA linking moiety;
Providing a second targeting agent wherein said second targeting comprises i) a second binding moiety that has a binding affinity to a site of the same said biological target which is different from the first site and ii) a second PNA linking moiety, wherein said first and second PNA linking moieties are at least partially complementary to each other to be able to hybridize and wherein the first binding moiety and the second binding moiety are able to interact cooperatively with the said biological target;
Contacting said first targeting agent, said second targeting agent and said biological target to form a supramolecular assembly of the first and second targeting agents which are assembled through the hybridization of their complementary PNA moieties and wherein said supramolecular assembly is bound to the biological target at both the first and the second sites of said target through the first and second binding moieties.

Another aspect of the invention provides a supramolecular assembly comprising:
a first targeting agent wherein said first targeting agent comprises i) a first binding moiety that has a binding affinity to a first site of a biological target and ii) a first PNA linking moiety;
a second targeting agent wherein said second targeting comprises i) a second binding moiety that has a binding affinity to a site of the same said biological target which is different from the first site and ii) a second PNA linking moiety,
   wherein said first and second PNA linking moieties are hybridized together through a hybridizing clamp formed by the hybridization of the parts of their PNA sequences which are complementary in presence of said biological target and wherein the first binding moiety and the second binding moiety are able to interact cooperatively with the said biological target.

Another aspect of the invention provides a pharmaceutical composition comprising:
a first targeting agent wherein said first targeting agent comprises i) a first binding moiety that has a binding affinity to a first site of a biological target and ii) a first PNA linking moiety;
a second targeting agent wherein said second targeting comprises i) a second binding moiety that has a binding affinity to a site of the same said biological target which is different from the first site and ii) a second PNA linking moiety, wherein said first and second PNA linking moieties are at least partially complementary to each other to be able to hybridize and wherein the first binding moiety and the second binding moiety are able to interact cooperatively with the said biological target,
as well pharmaceutically acceptable salts thereof and a pharmaceutically acceptable carrier, diluent or excipient thereof.

Another aspect of the invention is a process for the preparation of a supramolecular assembly according to the invention as defined below.

Another aspect of the invention provides the use of a supramolecular assembly according to the invention or a formulation thereof as an anticoagulant, in particular for the preparation or storage of blood samples/plasma.

Further objects and advantageous aspects of the invention will be apparent from the claims and/or from the following detailed description of embodiments of the invention with reference to the annexed drawings. The compounds described herein have an activity that parallels affinity proteins such as monoclonal antibodies or affibodies but are prepared synthetically and thus are not limited to canonical proteogenic amino acids.

### BRIEF DESCRIPTION OF THE DRAWINGS

FIG. 1 is a schematic representation of the design of supramolecular assemblies (6) of the invention with on-demand reversibility. a. The assembly of the supramolecular assembly (6) is catalysed by the binding to a biological target (4) (e.g. thrombin) which creates a highly potent and highly selective inhibitor from two initial compounds (1 and 2) with low potency and selectivity. The inhibition of the biological target can be rapidly reversed by addition of an antidote (7). b. Legend of components represented in a.

FIG. 2 represents the schematic and detailed chemical structures of the main compounds A1, A8 and E1 and the PNA antidotes used in the method of the invention as described in Example 1. a. Active site-directed inhibitor A1 comprising a first binding moiety 1a) that has a binding affinity to a first site of a biological target and a first PNA linking moiety 1b) (8-mer PNA); b. Exosite II-directed inhibitor E1, comprising a second binding moiety 2a) that has a binding affinity to a site of the same said biological target which is different from the first site and a second PNA linking moiety 2b) (complementary 8-mer PNA) wherein said first and second PNA linking moieties are at least partially complementary to each other to be able to hybridize and wherein the first binding moiety and the second binding moiety are able to interact cooperatively with the said biological target; c. Active site-directed inhibitor A8 which comprises A1 with a 4-mer toehold PNA (1c) linked to the first PNA linking moiety 1b); d. 8-mer antidote AD1; e. 12-mer antidote AD2; f to o: structure of binding moieties A2-A7 and E2-E23.

FIG. 3 represents the various design of supramolecular assemblies and structure activity relationship study (SAR) as described in Example 1. The inhibitors disclosed in this study are comprised of two fragments: the active site-directed fragments numbered A1 to A8 and the exosite II-directed fragment which are numbered E1 to E23. The combination of the two fragments yields a potent inhibitor named as the combination of the two assembled fragments (e.g., A1-E1 is the combination of active site fragment A1 and exosite II fragment E1). a. Thrombin inhibition data for the combined inhibitor versus the two fragments alone. b. Selectivity data for A1-E1 against a panel of common proteases. c. Effect of PNA length on inhibition. d. SAR data of the exosite II binder by varying charge. e. SAR data of the exosite II binder by varying hydrophobic amino acids instead of isoleucine—discovered as an important residue by performing an Ala scan of the peptide region (A1-E17, A1-E18, A1-E19, A1-E20, A1-E21, A1-E22).

FIG. 4 presents the results of in vitro and ex vivo evaluation of the activity a supramolecular assembly A1-E1 as described in Example 2. a. Fibrinogen inhibition assay of compounds A1-E1 and A1 and E1 alone. b. In vitro aPTT of A1-E1 in human (top) and mouse (bottom) plasma. c. Ex-vivo aPTT of A1-E1 at 0.314 μmol/kg (2.5 mg/kg) and 0.627 μmol/kg (5 mg/kg) versus Argatroban at 1.966 μmol/kg (1 mg/kg).

FIG. 5 presents the in-vivo inhibition of thrombin by the supramolecular assembly of the invention as described in Example 3. a. Time course of fibrin fluorescence intensity and total thrombus volume (left), and average fibrin intensity and average thrombus volume for control group (n=7), argatroban treated cohort (n=4, 2 mg/mL bolus followed by 12 mg/kg infusion) and A1-E1 treated cohort (n=5, 5 mg/kg bolus). b. Exemplar image of thrombus 15 minutes after needle injury without inhibitor (left), with argatroban (centre) and with A1-E1 (right). Platelets and fibrin are shown and collagen in the background in white, scale bar is 10 m.

FIG. 6 presents the reversal of thrombin inhibition induced by the supramolecular assembly of the invention by the addition of a competing oligonucleotide (antidote) as described in Example 4. a. Schematic representation of the antidote (AD2) addition to a supramolecular assembly A8-E1 bound to thrombin, resulting in the dislocation of the supramolecular assembly A8-E1 leading to the reversal of inhibition. b. Chemical structures of adenine and diaminopurine forming hydrogen bonds with thymine. c. Fluorogenic assay data showing the reversal of thrombin inhibition by addition of different concentrations of antidote after 30 minutes of inhibition. d. Fibrinogen assay data showing the reversal of thrombin inhibition by addition of antidote (1 eq.) after 30 minutes of inhibition. e. Calibrated Automated Thrombogram (CAT) of A8-E1 with and without antidote. f. Average fibrin intensity and average thrombus volume for control group (n=7), argatroban treated cohort (n=4, 2 mg/mL bolus followed by 12 mg/kg infusion), A8-E1 treated cohort (n=3, 5 mg/kg bolus) and A8-E1+AD2 treated cohort (n=3, 5 mg/kg+5 molar eq. antidote). g. Exemplar image of thrombus 15 minutes after needle injury without inhibitor (left), with A8-E1 (centre) and with A8-E1+AD2 (right). Platelets and fibrin are shown and collagen in the background in white, scale bar is 10 m.

### DETAILED DESCRIPTION OF THE INVENTION

The expression “interact cooperatively with a biological target” refers to agents that bind synergistically to biological target (e.g. a protein) of interest. According to a particular embodiment, those agents are characterized by both a binding and an effect which is synergistic: individually, they bind poorly but together they bind strongly which then translates into a synergistic effect on the target.

The identification of those agents can be achieved via DNA-encoded libraries using dual-display for new targets lacking prior information (Wichert et al., 2015, Nat. Chem., 7, 241-249; https://doi.org:10.1038/Nchem.2158 and Vummidi et al., 2022, Nat. Chem., 14, 141-152 (2022); https://doi.org:10.1038/s41557-021-00829-5). The same approach can also be considered with Fab fragments of antibodies (Kazane et al., 2013, J. Am. Chem. Soc., 135, 340-346; https://doi.org:10.1021/ja309505c).

The term “PNA linking moiety” refers to a molecular moiety comprising a peptide nucleic acid sequence from about 4 to 12 nucleic acids and can be of the same or different length on each targeting agent. The PNA sequence of one targeting agent only needs to be able to hybridize to a portion of the opposing PNA of the other targeting agent and not necessarily over its entire length. According to a particular embodiment, the oligonucleotide sequences which can be used as PNA linking moiety can be PNA sequences or known analogues (preferably D stereochemistry to avoid hybridization and cross talk with endogenous oligonucleotides), DNA or modified RNA for metabolic stability (preferably L stereochemistry to avoid hybridization and cross talk with endogenous oligonucleotides), other oligonucleotides such as locked nucleic acid or other well-known xenonucleic acids or PNA/DNA chimeras. The PNA linking moiety may further comprise modifications on the backbone to optimize physicochemical properties (Saarbach et al., https://doi.org/10.1016/j.cbpa.2019.06.006). The PNA linking moiety can be conjugated to an amino acid from the binding moiety of the targeting agent through amide bond reaction or various ligation chemistry strategies, for example by click reactions such as CuAAC, strain promoted azide-alkyne cycloaddition (SPAAC) or other click cycloadditions such as tetrazene-strained alkyne, cysteine addition to aldehyde, nitrile, Michael acceptor, substitution reaction based on the unique reactivity of thiol and benzyl, allylic or acetyl leaving groups.

The amino acids of the binding moiety of the targeting agent can be D or L or a combination thereof but should not compromise the binding of the targeting agent to the target. The binding moieties can incorporate non-natural amino acids or other chemical building blocks which are not amino acids.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease.

The term “efficacy” of a treatment according to the invention can be measured based on changes in the course of a disease in response to a use or a method according to the invention.

The term “subject” as used herein, refers to a human or a non-human mammal, such as non-human primate (e.g. chimpanzees and other apes and monkey species), a farm animal (e.g. cattle, sheep, pigs, goats and horses), a domestic mammal (e.g. dogs and cats), or a laboratory animal (e.g. rodents, such as mice, rats and guinea pigs).

The term “efficacy” of a treatment according to the invention can be measured based on changes in the course of disease in response to a use or a method according to the invention. For example, the efficacy of a treatment according to the invention can be measured by its impact on signs or symptoms of illness. A response is achieved when the subject experiences partial or total alleviation, or reduction of unwanted symptoms of illness. According to a particular embodiment, the efficacy can be measured through the assessment of tumor volume.

The term “pharmaceutical formulation” refers to preparations which are in such a form as to permit biological activity of the active ingredient(s) to be unequivocally effective and which contain no additional component which would be toxic to subjects to which the said formulation would be administered.

### Method of the Invention

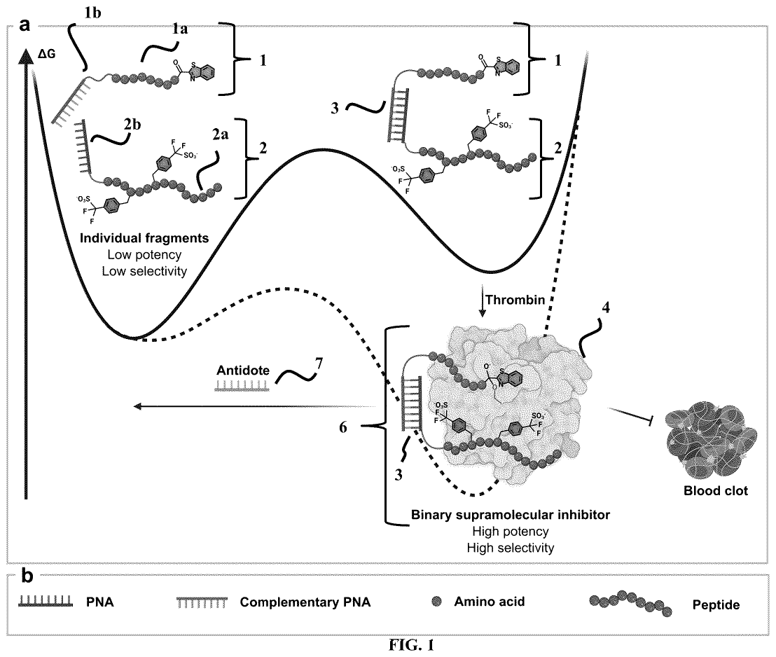

Referring to the figures, in particular FIG. 1a, is provided an illustration of a method of preventing or treating a disorder in a subject in need thereof by a supramolecular assembly (6) which activity can be reversed, said method comprising the steps of:
Providing a first targeting agent (1) wherein said first targeting agent comprises i) a first binding moiety (1a) that has a binding affinity to a first site of a biological target (4) and ii) a first PNA linking moiety (1b);
Providing a second targeting agent (2) wherein said second targeting comprises i) a second binding moiety (2a) that has a binding affinity to a site of the same said biological target (4) which is different from the first site and ii) a second PNA linking moiety (2b), wherein said first and second PNA linking moieties are at least partially complementary to each other to be able to hybridize and wherein the first binding moiety and the second binding moiety are able to interact cooperatively with the said biological target;
Contacting said first targeting agent and said second targeting agent with said biological target (4) to form a supramolecular assembly (6) of the first and second targeting agents which are assembled through the hybridization of their complementary PMA moieties into a hybridizing clamp (3) and wherein said supramolecular assembly (6) is bound to the biological target at both the first and the second sites of said target through the first and second binding moieties.

According to another embodiment, is provided a method of preventing or treating a thrombotic event in a subject in need thereof, said method comprising the steps of:
administering a combination of:
i) a first targeting agent wherein said first targeting agent comprises i) a first binding moiety that has a binding affinity to a first site of thrombin and ii) a first PNA linking moiety;
ii) a second targeting agent wherein said second targeting comprises i) a second binding moiety that has a binding affinity to a site of thrombin which is different from the first site and ii) a second PNA linking moiety, wherein said first and second PNA linking moieties are at least partially complementary to each other to be able to hybridize and wherein the first binding moiety and the second binding moiety are able to interact cooperatively with thrombin;
and wherein said first and second targeting agents are able to form a supramolecular assembly through the hybridization of their complementary PNA moieties and wherein said supramolecular assembly.

According to a particular embodiment, the first targeting agent is selected from A1 to A8.

According to another further particular embodiment, the second targeting agent is selected from E1 to E23.

According to a particular embodiment, the said first and said second targeting agents are contacted with the said target as a mixture or, when contacted separately, the said first and said second targeting agents are contacted with the target at similar times (little to no interval between the provision of each of those).

According to a particular embodiment, the above method is achieved through the administering of the said first and said second targeting agents into a subject in need thereof either as a mixture or separately but at similar times (little to no interval between the administration each of those).

According to a particular embodiment, when the said first and said second targeting agents are administered as a mixture.

According to a particular embodiment, when the said first and said second targeting agents are administered separately and the interval of administration between the two targeting agents will depend on their PK/PD profiles: if those are significantly different, the two agents could be dosed separately and at different time intervals and/or frequencies (e.g. one agent could be administered hourly and the other one daily if their half-life would allow such interval).

According to a particular embodiment, when the said first and said second targeting agents are administered as a mixture, the mixture contains free forms of said agents and a supramolecular assembly according to the invention and the equilibrium will then be shifted to the supramolecular assembly form in the presence of the target.

According to a particular embodiment, the said first and said second targeting agents are combined in a 1:1 ratio when administered.

According to a further embodiment, the method further comprises a step of administering an antidote agent (7) to reverse the activity of the supramolecular assembly (6), wherein said antidote agent is a competing oligonucleotide (antidote) comprising a sequence being complementary to at least a part of the first or the second PNA linking moiety. Hybridization with the competing oligonucleotide (antidote) would disrupt the hybridizing clamp leading to the disruption of the supramolecular assembly and to removal or weaker binding to the target.

According to a further particular embodiment, the antidote agent is a competitor PNA which is able to remove the hybridizing clamp due to its higher affinity with one of the PNA linking moiety and ability to hybridize with such said PNA linking moiety.

According to a further particular embodiment, the competitor PNA comprises 4-16 nucleobases (e.g. 8 to 12).

Another aspect of the invention is a method for preventing or treating a thrombotic event in a subject in need thereof, said method comprising administering in a subject in need thereof, a pharmaceutical formulation according to the invention comprising the precursors of a supramolecular assembly according to the invention and wherein said supramolecular assembly is formed in vivo in the presence of the target.

Another aspect of the invention is a method for preventing or treating a thrombotic event further comprising the administration of antidote agent.

According to another aspect of the invention, an antidote agent. is selected from AD1 and AD2.

Another aspect of the invention is a method for preventing or treating a thrombotic event in a subject in need thereof, said method comprising administering a supramolecular assembly according to the invention or a pharmaceutical formulation thereof in a subject in need thereof.

Another aspect of the invention provides the use of a supramolecular assembly according to the invention or a formulation thereof as an anticoagulant, in particular for the preparation or storage of store/prepare blood samples/plasma. Said method may comprise the ex-vivo contacting of a supramolecular assembly according to the invention or a formulation thereof with blood or plasma samples.

Another aspect of the invention is a process for the preparation of a supramolecular assembly or complex thereof according to the invention as defined below.

### Compounds of the Invention

FIG. 1a, further provides an illustration a supramolecular assembly (6) comprising:
a first targeting agent (1) wherein said first targeting agent comprises i) a first binding moiety (1a) that has a binding affinity to a first site of a biological target (4) and ii) a first PNA linking moiety (1b);
a second targeting agent (2) wherein said second targeting comprises i) a second binding moiety (2a) that has a binding affinity to a site of the same said biological target (4) which is different from the first site and ii) a second PNA linking moiety (2b), wherein said first and second PNA linking moieties are hybridized together through a hybridizing clamp (3) formed by the hybridization of the parts of their PNA sequences which are complementary and wherein the first binding moiety and the second binding moiety are able to interact cooperatively with the said biological target.

According to a particular aspect, the formation of a supramolecular assembly according to the invention is catalyzed by the presence of said target.

According to another particular aspect, is provided a complex formed by a supramolecular assembly (6) bound to said biological target (4).

According to a particular aspect, the distance between the two binding moieties is from about 10 angstroms to about 100 angstroms within the supramolecular assembly.

According to another particular aspect, the PNA linking moieties comprise 4 to 12 nucleobases.

According to another particular aspect, the free-end of the PNA linking moieties is acetylated.

According to another particular aspect, the PNA linking moieties are selected from GCAGTTGT, ACAACTGC and GCGAGCAGTTGT (SEQ ID NO: 1).

According to another particular aspect, the PNA backbone is serine-modified in the gamma position.

According to one particular aspect, the sequence of one of the first and second PNA linking moieties comprises 1 to about 6 nucleobases (“toehold sequence”) (e.g. 4 nucleic acids) on its free side (i.e. not linked to the binding moiety) which are not complementary to the PNA sequence of the other targeting agent.

According to another particular aspect, the PNA linking moiety may further comprise an active agent (e.g. cytotoxic agent for targeted drug delivery, nucleic acids for antisense therapy) or a labeling moiety such as dye or radio nuclei for imaging application (ex. PET) or photodynamic therapy or localized radiation therapy or even an albumin binding functional group to increase half-life in vivo.

Another aspect of the invention, the first targeting agent is selected from A1 to A8.

According to one particular aspect, the first targeting agent is A1.

Another aspect of the invention, second targeting agent is selected from E1 to E23.

According to one particular aspect, the second targeting agent is E1.

According to one particular aspect, is provided a supramolecular assembly comprising a first targeting agent of the invention and a second targeting agent of the invention wherein said first and second PNA linking moieties are hybridized together through a hybridizing clamp formed by the hybridization of the parts of their PNA sequences which are complementary to each other and wherein the first binding moiety and the second binding moieties are able to interact cooperatively with thrombin (e.g. A1-E1).

According to a particular aspect, is provided an anticoagulant supramolecular assembly according to the invention.

According to a particular aspect, the distance between the two binding moieties in an anticoagulant supramolecular assembly according to the invention is from about 30 angstroms to about 40 angstroms within the supramolecular assembly.

Another aspect of the invention provides a pharmaceutical composition pharmaceutical composition comprising:
a first targeting agent wherein said first targeting agent comprises i) a first binding moiety that has a binding affinity to a first site of a biological target and ii) a first PNA linking moiety;
a second targeting agent wherein said second targeting comprises i) a second binding moiety that has a binding affinity to a site of the same said biological target which is different from the first site and ii) a second PNA linking moiety, wherein said first and second PNA linking moieties are at least partially complementary to each other to be able to hybridize and wherein the first binding moiety and the second binding moiety are able to interact cooperatively with the said biological target, as well pharmaceutically acceptable salts thereof and a pharmaceutically acceptable carrier, diluent or excipient thereof.

Another aspect of the invention provides a pharmaceutical composition of the invention further comprising a supramolecular assembly of the invention.

Another aspect of the invention is a process for the preparation of a supramolecular assembly according to the invention as defined below.

### Synthesis of Compounds of the Invention

The elements of the supramolecular assemblies of the invention can be prepared by standard techniques as described herein such as by solid phase peptide synthesis or in solution.

The elements of the supramolecular assemblies are synthesized as two individual molecules which are prepared, purified and stored individually.

To form a supramolecular assembly according to the invention, the targeting agents are simply combined in a 1:1 ratio and provided to the target (e.g. thrombin) where they will assemble into a supramolecular assembly around the said target. In vivo, the targeting agents will encounter the target in the blood and in vitro, thrombin could be added to the sample.

### Compositions

Pharmaceutical compositions of the invention can contain one or more compounds according to the invention and a pharmaceutically acceptable carrier, diluent or excipient thereof.

Compositions of this invention may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents.

Further materials as well as formulation processing techniques and th elike are set out in Remington: The Science & Practice of Pharmacy, 23^{rd} Edition, 2020, Ed. Adeboye Adejare, Academic Press, which is incorporated herein by reference.

According to a particular aspect, is provided a pharmaceutical composition comprising at least one compound according to the invention and a pharmaceutically acceptable carrier, diluent or excipient thereof.

### Mode of Administration

Compounds and compositions of this invention may be administered or delivered in any manner including, but not limited to subcutaneous injection or i.v. infusion or injection.

The dosage administered, as single or multiple doses, to an individual will vary depending upon a variety of factors, including pharmacokinetic properties, subject conditions and characteristics (sex, age, body weight, health, and size), extent of symptoms, concurrent treatments, frequency of treatment and the effect desired.

### Combination

According to one embodiment of the invention, the compounds according to the invention and pharmaceutical formulations thereof can be administered alone or in combination with a co-agent useful in the prevention and/or treatment of a disease.

According to one aspect, compounds of the invention can be administered in combination with at least one therapeutic molecule.

The invention encompasses the administration of a compound of the invention wherein the compound is administered to a subject prior to, simultaneously or sequentially with a therapeutic regimen or at least one co-agent. The compound according to the invention that is administered simultaneously with said at least one co-agent can be administered in the same or different compositions and in the same or different routes of administration.

### Subjects

In an embodiment, subjects according to the invention are suffering from a disorder.

In an embodiment, subjects according to the invention are suffering from a condition susceptible to lead to a thrombotic event.

In an embodiment, subjects according to the invention are suffering from a thrombotic disorder.

In a further particular embodiment, subjects according to the invention are subjects suffering from a disorder treatable by immunotherapy, such as cancer. For example, in the case of patients undergoing CAR-T-based cancer immunotherapy, a method according to the invention allows the provision of antidote to the CAR-T response which is highly desirable in case of occurrence of a cytokine storm or other adverse effects linked to immune overactivation caused by the CAR-T based treatment.

In a further particular embodiment, subjects according to the invention are subjects suffering from a disorder treatable by immunomodulation. For example, for patients undergoing treatments with immunosuppressors, a method according to the invention allows the provision of an antidote to reverse the action of the immunosuppressors which is highly desirable in case of a severe infection.

### Use According to the Invention

According to an embodiment, is provided a method of preventing or treating a disorder in a subject in need thereof by a supramolecular assembly according to the invention which activity can be reversed in vivo.

The invention provides compounds of the invention, compositions thereof and methods using the same useful in the prevention and/or treatment of a medical disorder, in particular thrombotic events or a disorder treatable by immunotherapy or immunomodulation.

Any additional elements of the invention are described in Dockerill et al., 2024, https://doi.org/10.1038/s41587-024-02209-z and bioRxiv preprint doi: https://doi.org/10.1101/2023.11.12.566735 which are incorporated herein by reference.

References cited herein are hereby incorporated by reference in their entirety. Such modifications are intended to fall within the scope of the appended claims. The invention having been described, the following examples are presented by way of illustration, and not limitation.

### Examples

### General Methods

Unless otherwise specified, all reagents and solvents for the organic synthesis were purchased from commercial sources and were used without further purification. HPLC purification was performed with an Agilent Technologies 1260 infinity HPLC using a ZORBAX 300SB-C18 column (9.4×250 mm). LC-MS spectra were recorded on a DIONEX Ultimate 3000 UHPLC with a Thermo LCQ Fleet Mass Spectrometer System using PINNACLE DB C18 column (1.9 μm, 50×2.1 mm) operated in positive mode. All the LC-MS spectra were measured by ESI. MALDI-TOF Mass spectra were measured using a Bruker Daltonics Autoflex spectrometer operated in positive mode. High-resolution mass spectra (HRMS) were obtained on a Xevo G2 Tof spectrometer (Ionization mode: ESI positive polarity; Mobile phase: MeOH 100 μl/min). Automated solid-phase synthesis was carried out on an Intavis AG Multipep RS instrument.

### Synthesis of PNA-Peptide Conjugates

Resin (5.0 mg) was swollen in DCM for 10 minutes and washed twice with DMF. Iterative cycles of amide coupling (Procedure 1), capping of the resin (Procedure 4), and deprotection of the protecting group (Procedure 2 or 3) were performed to synthesize the PNA probes. The compounds were deprotected and cleaved from the resin using Procedure 5 and finally purified using HPLC.

2-Chlorotrityl Chloride Resin Loading: 2-Chlorotrityl chloride resin (1.46 mmol/g loading) was swollen in dry DCM for 30 min, followed by washing with DCM+1% DIPEA (1×3 mL) and DCM (10×3 mL). A solution of Fmoc-Xaa-OH (0.7 mmol/g resin) and DIPEA (4 eq. relative to resin functionalization) in DCM (final concentration 0.125 M of amino acid) was added to the resin, which was shaken at room temperature for 16 h. The resin was then washed with DCM (5×3 mL), DMF (5×3 mL) and DCM (5×3 mL). The resin was then capped via treatment with 17:2:1 v/v/v DCM:MeOH:DIPEA (5 mL) for 40 mins at room temperature. The resin was then washed again with DCM (5×3 mL), DMF (5×3 mL) and DCM (5×3 mL) prior to further use.

Rink Amide Resin Loading: Nova PEG® Rink amide resin (0.44 mmol/g, NovaBiochem) was swollen in DCM for 10 minutes and washed twice with DMF. Standard amide coupling (Procedure 1) was performed, followed by capping of the resin (Procedure 4). The resin was then washed again with DCM (5×3 mL), DMF (5×3 mL) and DCM (5×3 mL) prior to further use.

Procedure 1 (P1): Amide coupling. The corresponding Fmoc protected PNA monomer (Chouikhi, et al., 2012, Chem. Eur. J., 18, 12698-12704, https://doi.org/10.1002/chem.201201337) or amino acid (4.0 equiv., 0.2M in NMP) was incubated for 5 minutes with HATU (3.5 equiv., 0.5 M in NMP) and base solution [DIPEA, 1.2M (4.0 equiv.) and 2,6-lutidine 1.8 M (6.0 equiv.) in NMP]. The mixture was then added to the corresponding resin. After 20 minutes, the mixture was filtered, the resin was washed with DMF, and a new premixed reaction solution was added to the resin and let react for another 20 minutes. Finally, the resin was washed with 2×DMF, 2×DCM, and 2×DMF.

Procedure 2 (P2): Fmoc deprotection. A solution of 20% (v/v) piperidine in DMF was added to the resin and allowed to react for 5 minutes. The mixture was then filtered, the resin washed with DMF, and the sequence repeated for another 5 minutes. Finally, the resin was washed with 2×DMF, 2×DCM, and 2×DMF.

Procedure 3 (P3): Mtt deprotection. A solution (made from 244 mg of HOBt in 10 mL HFIP and 10 mL DCE) was added to the prewashed resin to reach a volume of 10 mL/g of resin and allowed to react for 5 minutes. The solution was flushed, the resin washed with DCM, and the sequence repeated for another 5 minutes. Finally, the resin was washed with 2×DCM and 2×DMF.

Procedure 4 (P4): Capping. The resin was treated with a capping mixture (0.92 mL acetic anhydride and 1.3 mL 2.6 lutidine in 18 mL DMF: 10 mL of solution/g of resin) for 5 minutes. After flushing the solution, the resin was washed with 2×DMF, 2×DCM, and 2×DMF.

Procedure 5 (P5): Cleavage from the resin and final deprotection. Resin (5.0 mg, 1.0 mol) was treated with 125 μL of a mixture of TFA and scavengers (440 μL TFA+25 mg phenol+25 μL water+10 μL triisopropylsilane) for 2 h. The resin was filtered, washed with TFA (50 L), and the collected fractions of cleavage product precipitated in cold ether (1.5 mL). After centrifugation, the pellet was vortexed again with cold Et₂O (1.5 mL) and centrifuged (14 000 rpm). The pellet was dissolved in H₂O/CH₃CN (3:1, 1.5 mL) and lyophilized to obtain a white powder.

Procedure 6 (P6): Microcleavage for quality control. The minimum number of beads were picked up with a pipette plastic tip and transferred to 50 μL of TFA. The solution was left for 1 h and transferred to 1.0 mL of ether. The ether solution was kept for 5 minutes at −20° C. and then centrifuged for 5 minutes at 14 000 rpm. The ether supernatant was removed, and the pellet dissolved in 20 μL 1:1 acetonitrile/water, which was then used to analyze by MALDI and/or LC-MS.

Procedure 7 (P7): On-resin Copper(I)-catalyzed azide-alkyne cycloaddition (CuAAC). To TBTA (2.0 mg) in 20 μL DMF was added 15 μL CuSO₄ (64 mg/mL in H₂O) followed by 50 μL of NaAsc (396 mg/mL in H₂O). Azide-containing peptide (2 eq. in 60 μL DMF) was added to the mixture which was mixed prior to the addition to 5.0 mg alkyne derivatized rink amide resin (0.0022 mmol). After 16 h of shaking, the mixture was filtered and the resin was washed with 6×250 μL of sodium diethyl dithiocarbamate 0.02 M in DMF, 6×250 μL of DMF, 6×MeOH and 6×DCM.

Procedure 8 (P8): Coupling of Fmoc-L-F₂Smp(nP)-OH. Fmoc-L-F₂Smp(nP)-OH was prepared following as previously described (Dowman, et al., 2021, Chem. Commun., 57, 10923-10926, https://doi.org/10.1039/d1cc04742f).

A mixture of Fmoc-L-F₂Smp(nP)-OH (0.003 mmol, 1.5 equiv.), HOBt (0.003 mmol, 1.5 equiv.) and DIC (0.003 mmol, 1.5 equiv.) were added to the corresponding resin and shaken overnight. The mixture was filtered and the resin was washed with 2×DMF, 2×DCM, and 2×DMF.

Procedure 9 (P9): Coupling of Arg(Pbf)-Benzothiazole. To 5.0 mg of resin (0.0022 mmol), Arg(Pbf)-Benzothiazole (synthesis described in https://doi.org/10.1038/s41587-024-02209-z) (0.0044 mmol, 2 equiv.) and HATU (0.0034 mmol, 1.5 equiv.) in NMP (100 L) were added followed by DIPEA (0.012 mmol, 6 equiv.). The reaction was shaken for 2 h. The mixture was filtered, and the resin was washed with 2×DMF, 2×DCM, and 2×DMF.

Procedure 10 (P10): Neopentyl deprotection. The precipitate collected after cleavage and ether precipitation was lyophilized. The remaining solid was dissolved in a solution 1M NH₄Ac and 6M GnHCl and shaken at 37° C. for 2 h. The solution was then diluted with H₂O/ACN (50:50) and purified by HPLC.

Characterization of PNA-peptide conjugates: Characterization of the PNA-peptide conjugates was done by MALDI (Bruker Daltonics Autoflex spectrometer with Flex control 3.4 software and analysis with FlexAnalysis 3.4) and/or LC-MS (DIONEX Ultimate 3000 UHPLC with a Thermo LCQ Fleet Mass Spectrometer System using PINNACLE DB C18 column (1.9 μm, 50×2.1 mm) with Thermo Xcalibur 2.2.SP1.48 software and analysis with Thermo Xcalibur Qual Browser 2.2.Sp1.48). For MALDI analysis, 1.0 μL of the sample (in either water or water/acetonitrile 1:1) was mixed with 1.0 μL of DHB matrix solution (30 mg of DHB in 1.0 mL of 70:30:0.01 water/acetonitrile/TFA), and the mixture spotted on a MALDI plate. The measurements were done in a positive linear mode. For LC-MS analysis, 20 μL of sample in water or water/acetonitrile 1:1 was injected on the LC and further analyzed by MS on a positive mode. Compounds containing the benzene disulfonic acid motif could only be analyzed by LC-MS due to a fragmentation when analyzed by MALDI.

### Thrombin Inhibition Assay

In vitro Inhibition of Human α-Thrombin. The inhibition of the activity of human α-thrombin (Haematologic Technologies, HCT0020) was followed spectrophotometrically using phe-Pro-Arg-Coumarin (synthesis described previously) as chromogenic substrate.

Inhibition assays were performed using 0.2 nM enzyme, 20 μM substrate, and increasing concentrations of inhibitor. The concentration of each inhibitor variant was determined using the absorbance of the PNA at 260 nm, measured by nanodrop. All reactions were carried out at 37° C. in 50 mM Tris-HCl pH 8.0, 50 mM NaCl, 1 mg/mL BSA in black 96-well microtiter plates (ref 267342, ThermoFisher). Reaction progress was monitored by excitation at 339 nm and emission at 439 nm using a SpectraMax or Tecan Spark Plate Reader. Dose-response curves were used to determine the IC₅₀ values using Prism 8.0 (GraphPad Software). For each inhibitor, the reactions were performed in triplicate, together with control reactions in the absence of enzyme. The initial velocity was calculated from the slope of the first 10 minutes of the assay. The curves were normalised to the well without inhibitor, where the initial velocity was set to 100% activity.

In the case of the antidote assay, the plate was removed from the plate reader at the desired time of addition (usually 30 minutes), 1 μL of antidote (100×) was added and reading was resumed.

### Fibrinogen Assay

Human α-thrombin (Haematologic Technologies, HCT0020, final concentration 2.5 nM) was incubated with compound (final concentration 15 nM) at 37° C. for 30 minutes. Fibrinogen (final concentration 1 mg/mL) was added and absorbance at 288 nm was measured using a SpectraMax Plate Reader. All reactions were carried out at 37° C. in 50 mM Tris-HCl pH 8.0, 50 mM NaCl, 1 mg/mL BSA in clear 96-well microtiter plates (Greiner Bio-One, 650201).

In the case of the antidote assay, the plate was removed from the plate reader at the desired time of addition (usually 30 minutes), 1 μL of antidote (100×) was added and reading was resumed.

### Selectivity Assays

The inhibition activity of A1-E1 was tested against human FIIa, FXIa, and FXa (Haematologic Technologies), αFXIIa and plasma kallikrein (PK) (Enzyme Research Laboratories). Chromogenic assays were followed spectrophotometrically using specific substrates: 100 μM Tos-Gly-Pro-Arg-pNA (Chromozym TH; Roche) for FIIa; 500 μM Pyr-Pro-Arg-pNA (L-2145; Bachem) for FXIa; 500 μM Moc-D-Nle-Gly-Arg-pNA (L-1565; Bachem) for FXa; and 200 μM or 400 μM D-Pro-Phe-Arg-pNA (Cayman Chemical) for αFXIIa or PK, respectively. The assay buffers were: 50 mM Tris-HCl pH 8.0, 50 mM NaCl for FIIa (0.2 nM); PBS pH 7.4 for FXa (0.5 nM); 25 mM Tris-HCl pH 7.5, 100 mM NaCl, 5 mM CaCl₂) for FXa (0.5 nM); 20 mM HEPES pH 7.6, 150 mM NaCl, 0.1% (w/v) PEG 8000, 0.01% (v/v) Triton X-100 for αFXIIa (4 nM); and 50 mM Tris-HCl pH 8.0, 150 mM NaCl for PK (0.25 nM). Bovine serum albumin (Sigma) was added to all buffers at 1 g/L. All reactions were initiated by the addition of the protease and carried out at 37° C. in 96-well flat-bottom microtiter plates. Reaction progress was monitored at 405 nm for 30 minutes (60 minutes for FXa and αFXIIa), on a multi-mode microplate reader (Synergy2, BioTek) with measurements taken every 5 minutes. All measurements were performed in duplicate. IC₅₀ values were determined from the log-dose-response curves with Prism 9 (GraphPad Software).

### SPR Experiments

SPR experiments were performed on a Biacore T200 instrument (GE Healthcare) at 25° C. in PBS-P+ buffer (10×stock from Cytiva Life Sciences, 28995084). Biotin-PNA(8mer) was immobilised on a Streptavidin Series S sensor chip (Cytiva Life Sciences, 29104992). Prior to immobilisation, the two channels were conditioned with 1 M NaCl in 50 mM NaOH. After stabilisation, the compound (solution in PBS-P+) was flowed over one of the flow cells of the sensor chip at a concentration of 50 nM at a flow rate of 10 μL min⁻¹ with a response unit (RU) target of 500. Biotin-PNA(8mer) reached an RU value of 513.7. The system (not including the flow cells) was washed with 50% isopropanol in 1 M NaCl and 50 mM NaOH after each ligand injection. Kinetic measurements consisted of injections (association 400 s, dissociation 450 s, flow rate: 30 μL min⁻¹) of decreasing concentration of PNA (4, 6 and 8mer) (2-fold cascade dilutions from the starting concentration). The chip was regenerated between cycles by one injection of regeneration solution (50 mM NaOH) for 10 s at a flow rate of 20 μL min⁻¹, followed by a 10 s stabilisation period. Binding was measured as resonance units over time after blank subtraction, and the data interpreted using the Biacore T200 software, version 3.2. All measurements were performed in duplicate. The K_{D} values were calculated based on steady-state affinity (1:1 binding).

### Activated Partial Thromboplastin Time In Vitro

Activated Partial Thromboplastin time (aPTT) measurements were performed on a BFT II benchtop analyser using the manufacturer's instructions. Dade Actin™ FSL Activated PTT Reagent (Cat. No. 23-044-647) and calcium chloride solution (SMN/catalog number—10446232 ORHO37) were both sourced from Siemens Healthcare Diagnostics Products, GmbH and lyophilised pooled human reference plasma (Pooled Norm. Cat. No. 00539) was purchased from Diagnostica Stago, Australia and New Zealand Victoria. Pooled human plasma was reconstituted as per manufacturer's instructions (MQ, 30 minutes, RT). Pooled mouse plasma was prepared by collection of whole blood from 3-4 C57B16 mice (ABR, NSW) into sodium citrate (3.8%), with plasma isolated by centrifugation at 5,000×g for 15 minutes and stored on ice until required.

Human or mouse plasma was incubated with inhibitors at the indicated concentrations and pre-warmed to 37° C. Fifty L of each plasma/inhibitor mixture was incubated with Actin™ FSL (50 mL) in a stirred reaction vessel for 3 minutes, prior to addition of 50 mL calcium chloride solution, to initiate coagulation. The time taken for fibrin clot formation was recorded in a semi-automated fashion using the BFT II Analyzer which employs a turbo-densitometric detection technique.

### Ex-Vivo aPTT

All procedures involving the use of animals were performed as approved by the University of Sydney Animal Ethics Committee (USyd AEC, protocol 2021/1912). C57B16 mice (25-30 g) were anaesthetised using a mixture of ketamine (125 mg/kg) and xylazine (12.5 mg/kg) (intraperitoneal [i.p.] delivery), then, A1 and E1 fragments were administered as a single solution in a 1:1 molar ratio as a single bolus delivered intravenously via the femoral vein at either 2.5 or 5.0 mg/kg. The formation of an assembly A1-E1 is dynamic and reversible. The administered solution will be a mixture of individual fragments and assembled fragments (A1-E1). Once they interact with the target (thrombin), the assembly will be favoured. Blood was drawn from the IVC at the indicated times into citrate anticoagulant (3.8%), plasma isolated as described above for in vitro aPTT studies, and aPTT assessed via changes in plasma opacity at 405 nm using a ClarioSTAR plate reader fitted with dual injectors heated to 37° C., using a modified version of the aPTT protocol described above. Briefly, injectors were primed for Dade Actin™ FSL Activated PTT Reagent (Line A) and calcium chloride solution (line B), and mouse plasma aliquoted in duplicate (25 μL) into wells of a Nunc 368-well polystyrene plate (Cat. No. Z723010, Sigma-Aldrich). Following injection of 25 mL Dade Actin™ FSL, the plate was mixed using the orbital shaking function for 2 s (500 rpm) and incubated for 182 s at 37° C. At this time (designated t=0 s) 25 mL of calcium chloride solution was injected, the plate mixed as described above, and absorbance measurements taken at 405 nm for 360 intervals (22 flashes per well, 0.5 sec interval time). Clotting time was denoted by the timing of initial inflection point, denoting transition of plasma from transparent to opaque.

### Calibrated Automated Thrombogram (CAT)

Normal lyophilised human pooled plasma (Pool Norm #00539, Diagnostica Stago S.A.S., Asnières-sur-Seine, France) was reconstituted and incubated for 30 mins at 37° C. Vehicle and various inhibitor concentrations were incubated in plasma post 30-mins incubation. Thrombin assays were performed via a Hemker Calibrated Automated Thrombinoscope (Diagnostica Stago) using a Fluoroskan Ascent® plate reader (Thermo Scientific, MA, United States). All experiments were conducted in triplicate in 96-well microplates for fluorescence-based assays (M33089, Thermo Scientific, MA, United States) and calibrated using untreated plasma and a thrombin calibrator (#86192, Diagnostica Stago S.A.S., Asnieres-sur-Seine, France). Thrombinoscope experiments were conducted following patented commercial protocols: in brief, each sample well was filled 20 L PPP-reagent, containing a mixture of phospholipids and tissue factor (#86193, Diagnostica Stago S.A.S., Asnieres-sur-Seine, France). Eighty μL plasma (untreated/treated) was then added to each of these wells, mixed using reverse pipetting, and the well plate was incubated by the plate reader at 37° C. for 10 minutes. Meanwhile, a FluCa-Kit (#86197, Diagnostica Stago S.A.S., Asnieres-sur-Seine, France) containing Fluo-Buffer and Fluo-Substrate was warmed to 37° C. Following incubation, the thrombinoscope dispenser was flushed, emptied, and filled with a FluCa mixture of the Fluo-Buffer and Fluo-Substrate. Twenty L of the FluCa mixture were dispensed to each well containing plasma samples, commencing the coagulation reaction. Thrombin activity (nM) was measured over 1 h, with thrombogram parameters including lag time (mins), velocity index (nM/min), time to peak (mins), peak height (nM), endogenous thrombin potential (ETP) (nM*min), and time to tail (min).

### Needle Injury Thrombosis Model

C57Bl/6J mice were purchased from Australian BioResources (ABR, NSW, Australia) and housed at the Laboratory Animal Services (LAS) facility (the University of Sydney). All animals were housed in a 12-hour light/dark cycle with access to food and water ad libitum. For intravital mouse studies, male mice aged between 5-8 weeks old (15 g-20 g) were used. All studies were approved by the University of Sydney Animal Ethics Committee (Protocol 2021/1912) in accordance with the requirements of the Australian Code of Practice for the Care and Use of Animals for Scientific Purposes.

A clinical preparation of argatroban (Argatra/Exembol®) was purchased from Mitsubishi Tanabe Pharma (Germany) and prepared in sterile saline with 25% (vol/vol) of propylene glycol. Synthesized PNA inhibitors and PNA inhibitors+antidote solutions were prepared in sterile saline at a concentration of 2 mg/mL. Ketamine (150 mg/kg)- and xylazine (15 mg/kg)-anesthetized oxygen-supplemented C57BL/6J male mice (15-25 g) were subjected to an intravital needle-injury model, as previously described (Agten et al., 2021, Angew. Chem. Int. Ed. 60, 5348-5356, https://doi.org/10.1002/anie.202015127).

Systemic injection of a DyLight 647 anti-GPIbβ antibody (X647 Emfret, Germany, 100 μg/kg) and Alexa 546-anti fibrin antibody (0.31 μg/kg) was performed prior to vessel injury, to monitor thrombus formation and fibrin generation, respectively. Argatroban (80 ug/kg bolus; 40 μg/kg/min 60-minute infusion) was delivered via jugular catheter using a Harvard apparatus pump (Cat #704504; Pump Elite 11 I/W Single Syringe Pump, NSW, Australia). Injections of PNA inhibitors or PNA inhibitors+antidote (5 mg/kg bolus every 30 minutes) was delivered intravenously. 3-4 successive injuries were created in multiple vessels in each mouse from each treatment group. Following each injury, platelet thrombus formation and fibrin generation were monitored over a 15-minute period using a confocal intravital microscopy platform (Nikon AiR-si; objective: Apo LWD, ×40 magnification, 1.15 numerical aperture, water immersion; sequential excitation: 488-, 561-, and 638-nm lasers; emission: 525/50-, 595/50-, and 700/75-nm filters; using NIS Elements Advanced Research acquisition software). The microscope stage and objective were maintained at 37° C. throughout the experiment via a Peltier heater (OkoLab). Surface renders of confocal stacks representing thrombi from separate groups were generated using Imaris (Ver. 9.8 Bitplane AG, Zurich, Switzerland).

Quantitative analysis of thrombus volume over time: NIS-Elements software (Nikon, Japan) was used to apply a threshold to the DyLight 649-conjugated anti-mouse GP1bβ antibody signal for each xyz stack in a time series and was used to calculate the volume for each time point.

Quantitation of change in fibrin amount over time: The signal obtained from DyLight 649-conjugated anti-mouse GP1bβ antibody for each xyz stack in a time series was thresholded to create a mask. The total signal (AU) from the Alexa Fluor 546-conjugated anti-fibrin antibody within this mask (i.e., the fibrin signal within the thrombus) for each time point was then quantified using NIS-Elements software (Nikon, Japan).

Statistical analysis: Statistical significance between multiple treatment groups was analyzed using a RM one-way analysis of variance (ANOVA) with Bonferroni post-testing. Statistical significance between 2 treatment groups was analyzed using a paired Student t test with 2-tailed P values (Prism software; GraphPAD Software for Science, San Diego, CA). Data are presented as means±SEM where ‘n’ equals the number of independent experiments performed.

### Example 1: Synthesis and Characterization of Supramolecular Thrombin Inhibitors

In order to validate the design of supramolecular assemblies according to the invention (FIG. 1), the method of the invention was used for the design of thrombin inhibitors inspired by those produced naturally by blood feeding (hematophagous) organisms such as leeches, ticks, mosquitoes and flies that secrete small protein thrombin inhibitors in their salivary glands to facilitate acquisition and digestion of a bloodmeal. These salivary proteins exhibit potent thrombin inhibition by interacting with two distinct binding sites on thrombin but this activity cannot be easily reversed due to their extremely high affinity for thrombin.

Hyalomin 1 (Hya1), a 59-residue sulfated protein secreted by the tick Hyalomma marginatum rufipes that shares sequence similarity to other tick anticoagulants proteins, but is the most potent thrombin inhibitor in the family (Ki=5.4 pM) was chosen as a starting point.

By analysis of the X-ray crystal structures of several of these proteins complexed with thrombin (e.g., tick-derived madanin-1 (PDB 5L6N) (Thompson et al., 2017, Nat. Chem. 9, 909-917; https://doi.org:10.1038/Nchem.2744) and TTI from the tsetse fly (PDB 6TKG)) (Calisto et al., 2021, Cell Chem. Biol. 28, 26-33.e28) together with the thrombin inhibitory data, it was hypothesized that the potent inhibition exhibited by these molecules may be derived from interactions at two loci of thrombin, the active site and exosite II, separated by 20-30 Å.

For the peptide targeting exosite II, sequences from several salivary proteins from hematophagous organisms that possess sulfotyrosine residues as a common post-translational modification (PTM) that has been shown to enhance activity were investigated (Thompson et al., 2017, supra; Calisto et al., 2021, supra; Watson et al., 2019, Proc. Nat. Acad, Sci. USA 116, 13873-13878; https://doi.org:doi:10.1073/pnas.1905177116; Ripoll-Rozada et al., 2022, Biochem. Soc. Trans. 50, 387-401; https://doi.org:10.1042/Bst20210600).

Considering the reported lability of the tyrosine sulfate PTM, a synthetic analogue of the natural modification, namely (sulfono(difluoro)methyl-phenylalanine: F2Smp) was used. (Dowman et al., 2021, Chem. Commun. 57, 10923-10926; https://doi.org:10.1039/D1CC04742F).

For the link between the two binding motifs in the supramolecular anticoagulant assembly, the synthetic DNA mimetic PNA (Egholm et al., 1993, Nature 365, 566-568; https://doi.org:10.1038/365566a0) was chosen based on the tunability of the hybridisation dynamics of this molecular class to provide anticoagulant reversibility, its metabolic stability, and the compatibility of its chemistry with peptide synthesis (Barluenga et al., 2015, Acc. Chem. Res. 48, 1319-1331; https://doi.org:10.1021/acs.accounts.5b00109).

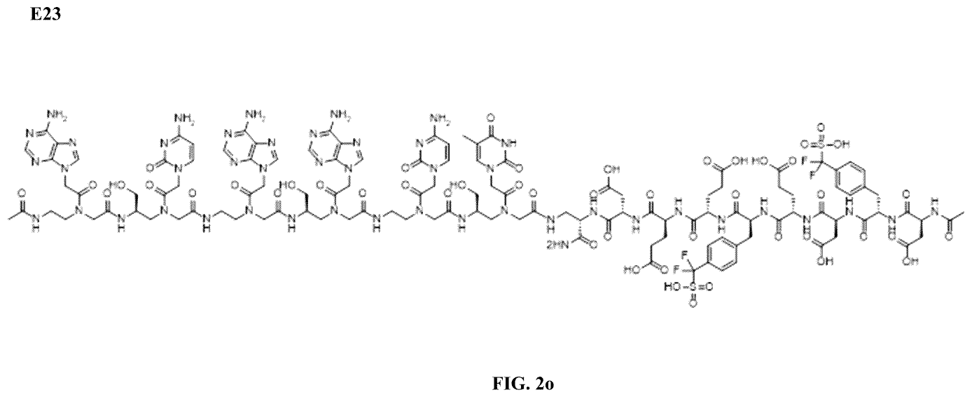

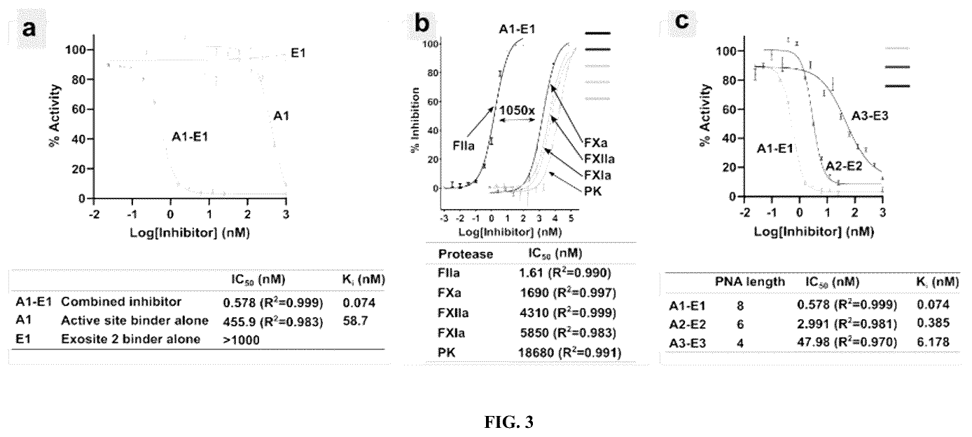

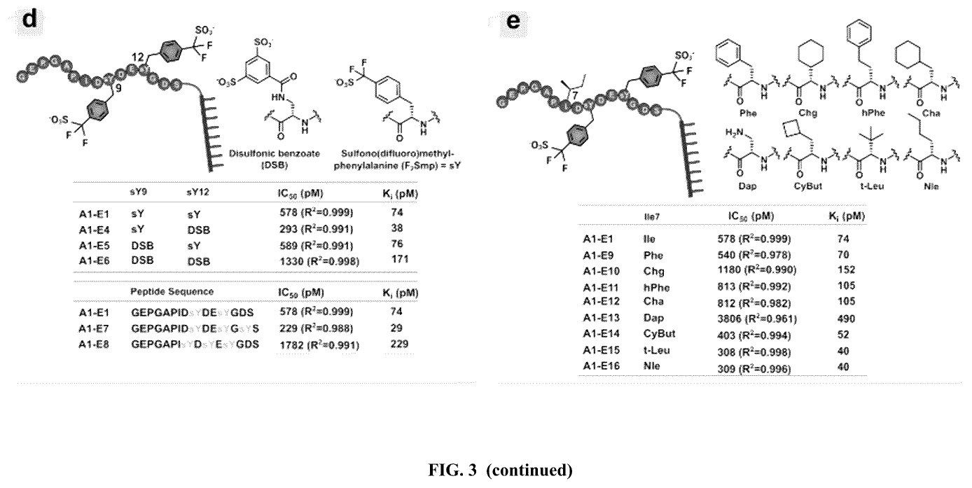

Solid-phase synthesis was used to prepare:
the first targeting agent (1), i.e. active site targeting peptide fragment A such as a targeting peptide fragment A1, A2 and A3 comprising a first binding moiety derived from Hya1 fused to a ketobenzothiazole warhead) (1a) linked to PNA linking moiety (1b), and
the second targeting agent (2), i.e. fragment E such as a targeting peptide fragment E1, E2 and E3 comprising a second binding moiety derived from the exosite II binding region of TTI) (2a) linked to a complementary PNA linking moiety (2b) (FIG. 3).

Given the known importance of two native negatively charged sulfotyrosine residues for interaction with the heparin-binding exosite II in TTI, incorporated two difluorosulfonomethylphenylalanine (F₂Smp) residues were incorporated as stable mimics in fragment E1. Fragment A1 showed moderate inhibitory activity against thrombin (Kᵢ 58.7 nM) in a fluorogenic thrombin-activity assay, while E1 alone possessed no inhibitory activity (FIG. 3a). However, an 800-fold enhancement of activity was observed when both components were added together, resulting from the use of the 8-mer PNA supramolecular connection, with the combination A1-E1 exhibiting a Kᵢ of 74 pM (FIG. 3a). Advantageously, this supramolecular inhibitor also gained selectivity for thrombin when tested against a panel of proteases present in the coagulation pathway including FXa, FXIa, FXIIa and PK (>1000-fold, FIG. 3b). It is noteworthy that, like thrombin, the substrate specificity for factor FXa and FXIa also strongly favour Arg at position 1 next to the cleavage site (P1) since these Serine proteases chop next to arginine (like thrombin) (Gosalia et al., 2005, Mol. Cell. Proteomics 4, 626-636; https://doi.org:10.1074/mcp.M500004-MCP200; Dementiev et al., 2018, Blood Adv. 2, 549-558; https://doi.org:10.1182/bloodadvances.2018016337) but only thrombin benefits from the bivalent interaction of the supramolecular drug, resulting in >1000-fold selectivity. To further investigate the supramolecular connectivity between the two fragments, the length of PNA from 8-mer was reduced to 6-mer (A2-E2) or 4-mer (A3-E3), while keeping the distance between the two binding fragments similar (˜30-40 Å). This led to a progressive decrease of activity (FIG. 3c). However, the assembly composed of the shortest supramolecular linker (4-mer: A3-E3) was still 10-fold more potent than the active site inhibitor alone (A1). Taken together, these data support a cooperative interplay between the supramolecular interaction of the PNA (A1 binding active site, E1 binding exosite II and A1 and E1 hybridising) and the inhibition of thrombin through engagement with both the active site and exosite II. The hybridisation K_{D} of the 4-mer PNA was measured by SPR to be 4.14 μM at 25° C., yet the supramolecular assembly still yields a benefit at concentrations well below its K_{D}. A cooperativity in the inhibition is observed if the equilibrium re-binding of the active site ligand is faster in the supramolecular assembly-enzyme complex than the dissociation of the supramolecular assembly. It stands to reason that the longer PNA with slower k_{off} yields better cooperativity.

The use of the PNA as a linking moiety also provides the opportunity to quickly assemble analogues and perform structure-activity studies since new combinations can be generated simply by mixing the binary ligands. These could include ligands from different blood sucking insect species (e.g. A1-E1 versus A1-E23 using the exosite II fragment from Hyalomin1 and Madanin1, respectively). Other stable sulfotyrosine mimics in the exosite II binding fragment were explored by substituting F₂Smp (sulfono(difluoromethyl)phenylalanine) for disulfonic benzoate (DSB) in positions 9 and 12 of the E fragment to generate variants of this binding moiety: E4 (sY12→DSB), E5 (sY9→DSB) and E6 (sY9,12→DSB) that could be used to form supramolecular assemblies with active site binding fragment A1 by simple mixing (FIG. 3d) (Calisto et al., 2021, Cell Chem. Biol. 28, 26-33.e28, https://doi.org/10.1016/j.chembiol.2020.10.002).

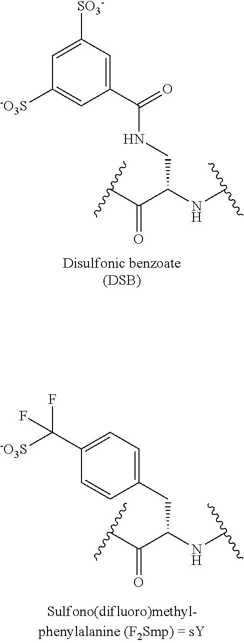

Inclusion of DSB in place of F2Smp at position 12 led to a two-fold gain of activity (A1-E4, FIG. 3d) but replacement of both F2Smp residues with DSB moieties led to a decrease of inhibition (A1-E6, FIG. 3d). The position and number of sulfotyrosine mimics also had a strong impact (A1-E1 vs A1-E7,8, FIG. 3d). Next, an alanine scan of the peptide sequence of E1 that targeted exosite II was performed (A1-E17, A1-E18, A1-E19, A1-E20, A1-E21, A1-E22). This revealed an isoleucine residue at position 7 (Ile7) as important, an observation consistent with the structure of the TTI-thrombin complex (PDB 6TKG) (Calisto et al., 2021, supra) which shows this isoleucine filling a hydrophobic pocket. A moderate (ca. 2-fold) gain in activity could be achieved with substitution for hydrophobic non-proteinogenic amino acids (e.g., t-Leu or Nle in A1-E15 or A1-E16, respectively, FIG. 3e). In this case, A1 was combined with E15 (wherein Ile is swapped for tLeu) and A1 was combined with E16 (wherein Ile is swapped for Nle).

### Example 2: In Vitro Evaluation of the Supramolecular Inhibitor

Having established the feasibility of the supramolecular inhibitor concept, A1-E1 was tested in biochemical assays and for anticoagulant activity in vitro. Towards this end, first, the inhibition of fibrinogen proteolysis, whereby A1-E1 exhibited complete inhibition at 100 nM (FIG. 4a) was investigated whilst A1 or E1 alone were comparable to no inhibitor. Having demonstrated that A1-E1 was able to prevent fibrinogen proteolysis in vitro, an activated partial thromboplastin time (aPTT) assay in both human and mouse plasma was used. aPTT assays are routine tests carried out by physicians and is an indicator of the function of coagulation factors in the intrinsic and common pathways, and effective inhibition of thrombin is expected to lengthen the time plasma takes to clot.

A clinically significant increase in clotting is said to be 2-fold and A1-E1 exhibited a therapeutically significant prolongation of clotting time in both human and mouse plasma at a concentration as low as 250 nM (FIG. 4b).

The effects of A1-E1 on thrombin generation in a calibrated automated thrombogram (CAT) were then investigated. The CAT employs a fluorogenic thrombin substrate, thus allowing measurement of thrombin formation in plasma in real time. This is of particular importance since thrombin generation is a dynamic process, the coagulation cascade has many feedback loops and inhibitory pathways that are all directly or indirectly influenced by the developing thrombin concentration and thrombin plays a central and pivotal role throughout the whole process. Additionally, and in contrast to aPTT assays, the CAT allows for a large variation in the concentration and character of the trigger used and can therefore be implemented to detect subtle differences between thrombin inhibitors. A1-E1 potently inhibited thrombin activity in both the initiation phase and propagation phase of coagulation and was able to completely inhibit thrombin activity at 2.5 μM.

### Example 3: In Vivo Evaluation of the Supramolecular Inhibitor

Having confirmed that the supramolecular anticoagulant assembly potently inhibited thrombin activity and possessed anticoagulant activity in vitro, it was investigated the efficacy of A1-E1 at inhibiting thrombus formation in vivo. To determine a suitable dose for the in vivo efficacy study, an ex vivo aPTT assay was used.

Briefly, A1-E1 was administered intravenously to mice at 2.5 or 5 mg/kg and blood samples were collected at 5, 15 and 45 mins. Clotting times were then measured using a standard aPTT protocol and showed that a single 5 mg/kg bolus was effective at prolonging the aPTT >2 fold for 30 mins (FIG. 4c).

The in vivo efficacy of the supramolecular anticoagulant A1-E1 was then tested compared to standard of care argatroban in a localised needle injury model (Watson et al., 2018, ACS Central Science 4, 468-476; https://doi.org:10.1021/acscentsci.7b00612).The injury leads to both fibrin formation and platelet aggregation in thrombus formation, which were visualized by Alexa-647 α-fibrin and Dylight-488 αGBP1bβ, respectively (Kaplan et al., 2015, Nat Commun 6; https://doi.org:ARTN 783510.1038/ncomms8835).

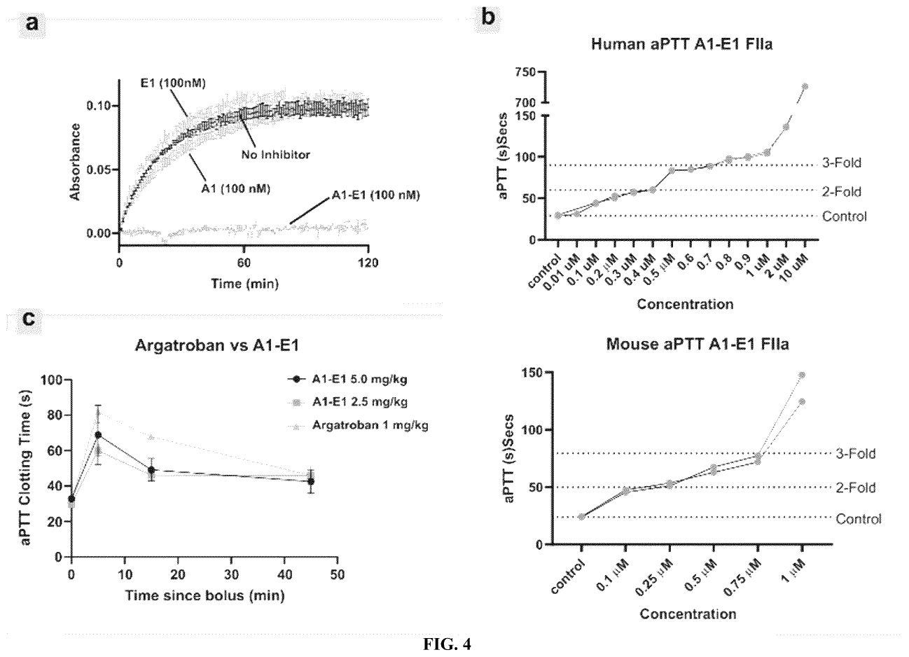

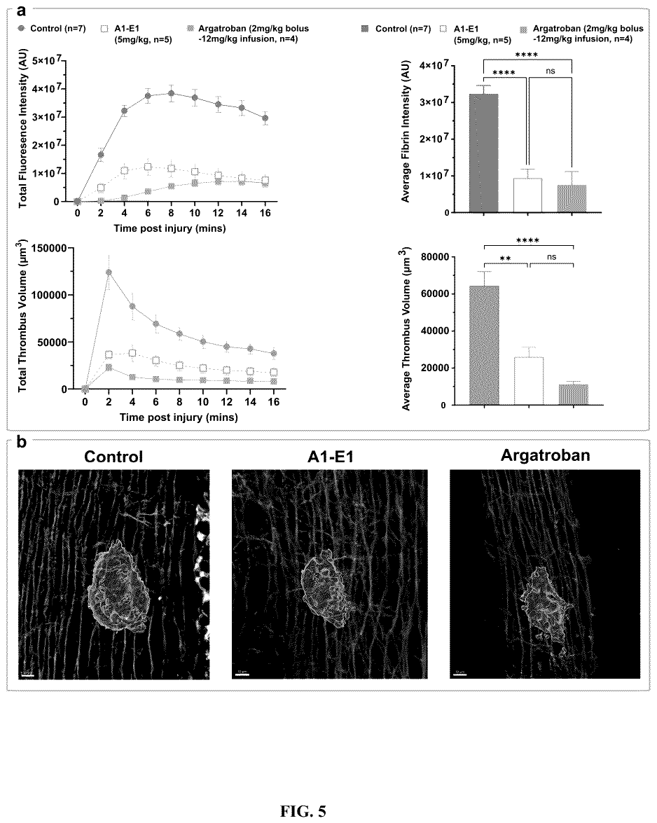

Owing to its short half-life in vivo, argatroban was dosed at 3.9 μmol/kg (2 mg/kg) IV bolus followed by an infusion at 24 μmol/kg over 60 minutes (12 mg/kg, total dose: 27.9 mol/kg). A1-E1 was dosed twice at 0.63 μmol/kg (5 mg/kg) IV bolus 30 minutes apart (total dose: 1.3 μmol/kg). Both A1-E1 and argatroban showed significant decrease in both fibrin formation and thrombus size (FIG. 5). Following treatment with the supramolecular anticoagulant A1-E1, followed by injury, near complete inhibition of fibrin deposition at the site of injury was observed when compared to control injuries (FIG. 5). Further, A1-E1 achieved a similar level of anticoagulation to a bolus infusion of argatroban at the 5 mg/kg dosing regimen (FIG. 5). On a molarity basis, A1-E1 yielded comparable results to the standard of care (argatroban) at 24-fold lower drug loading indicating that the potent inhibitory activity observed in vitro translates in vivo.

### Example 4: On-Demand Switching Off Inhibitory Activity In Vitro and In Vivo

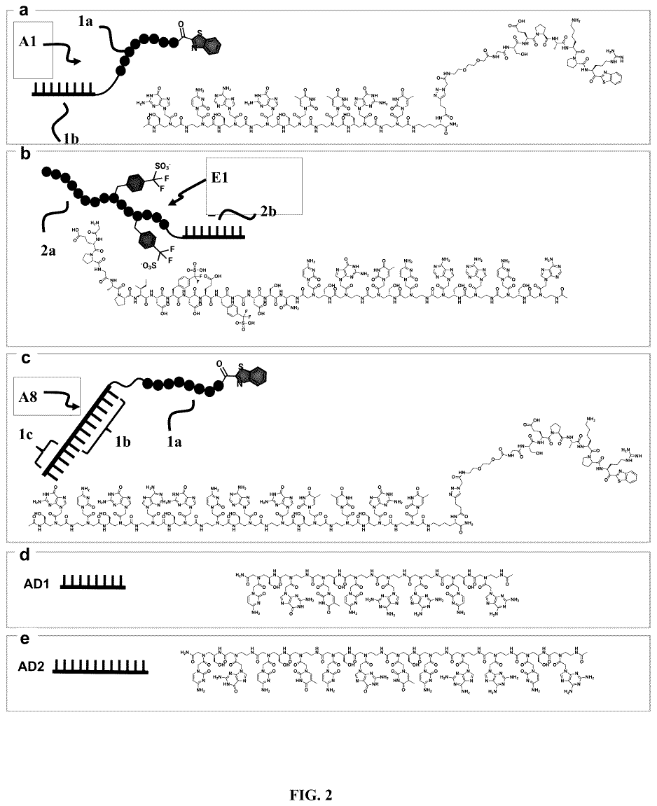

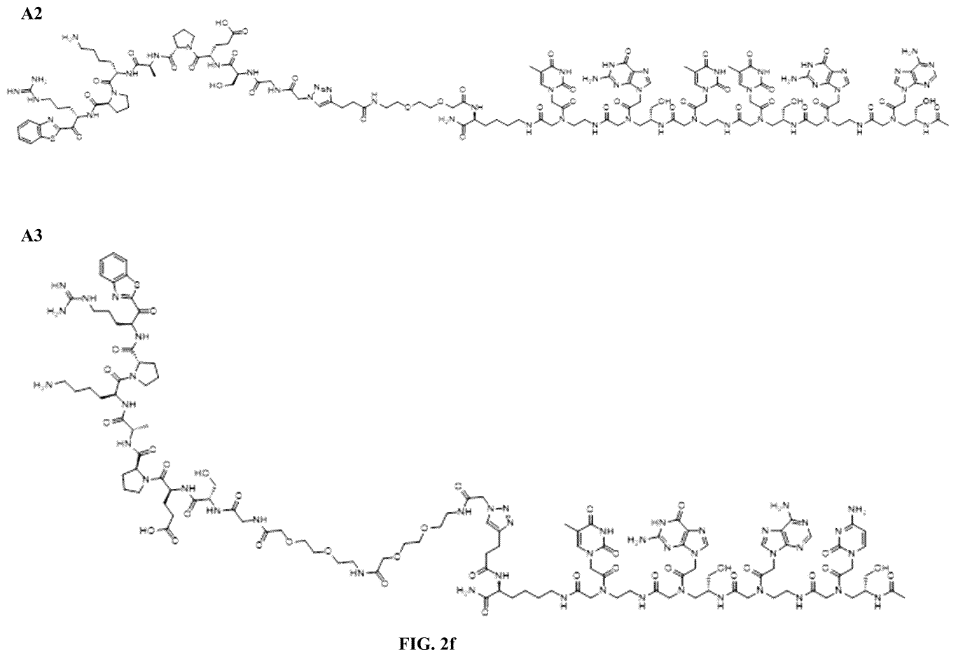

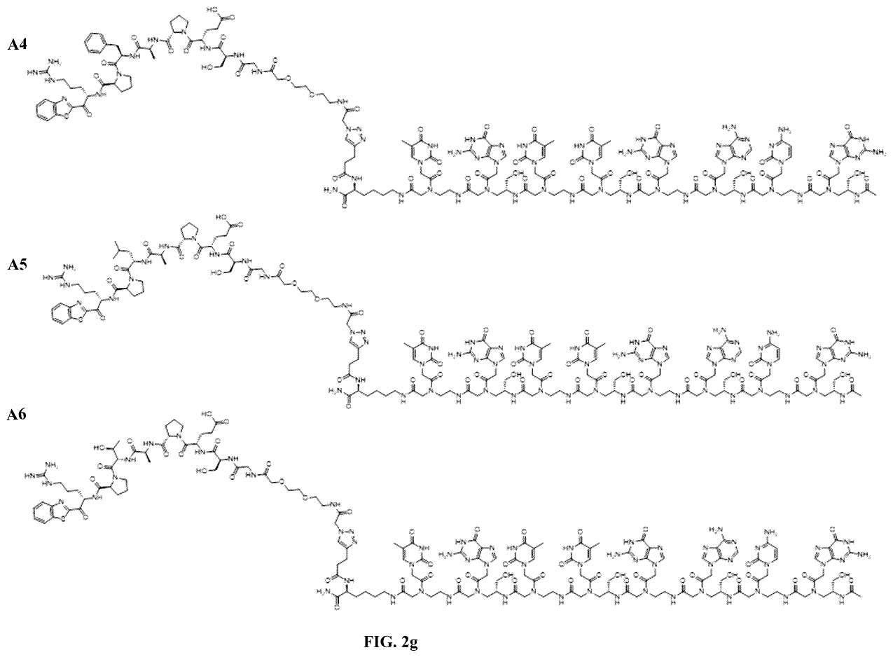

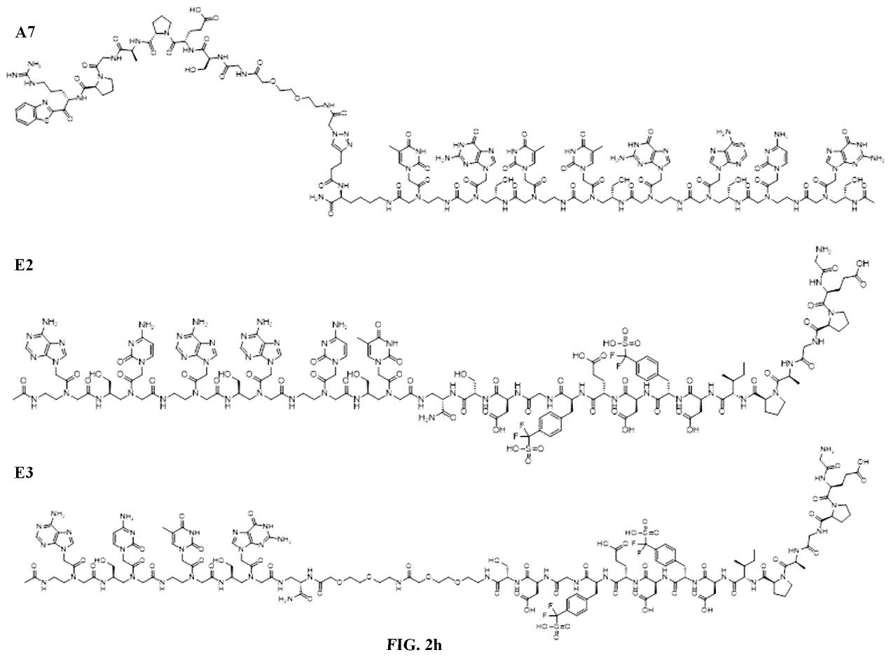

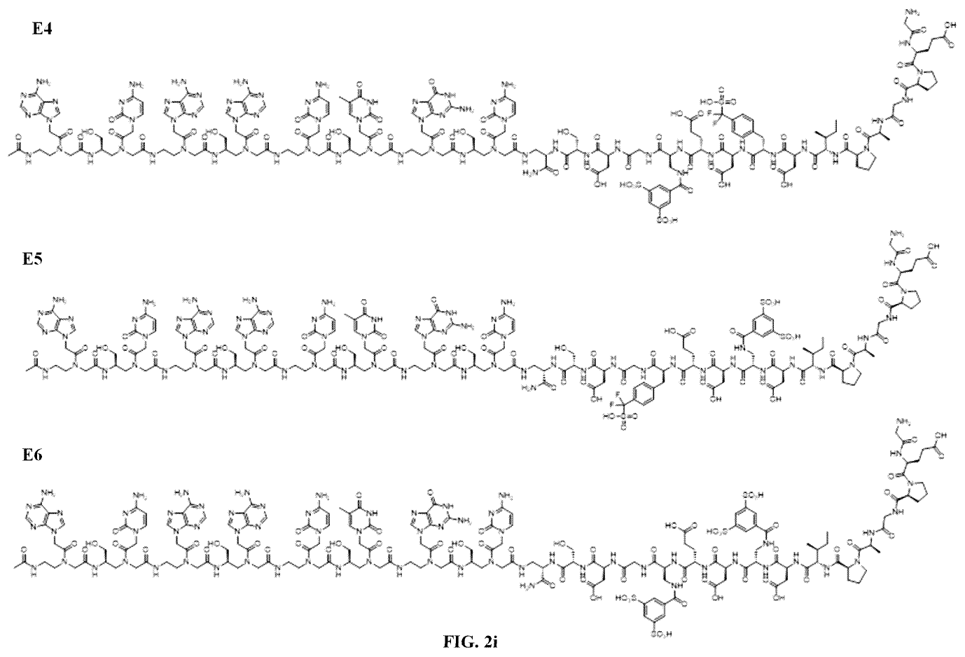

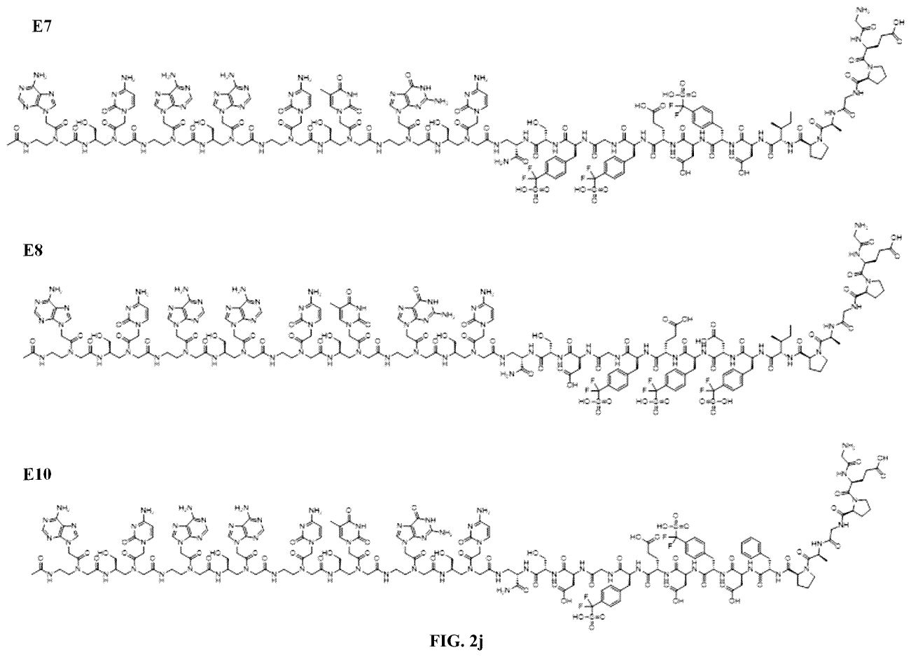

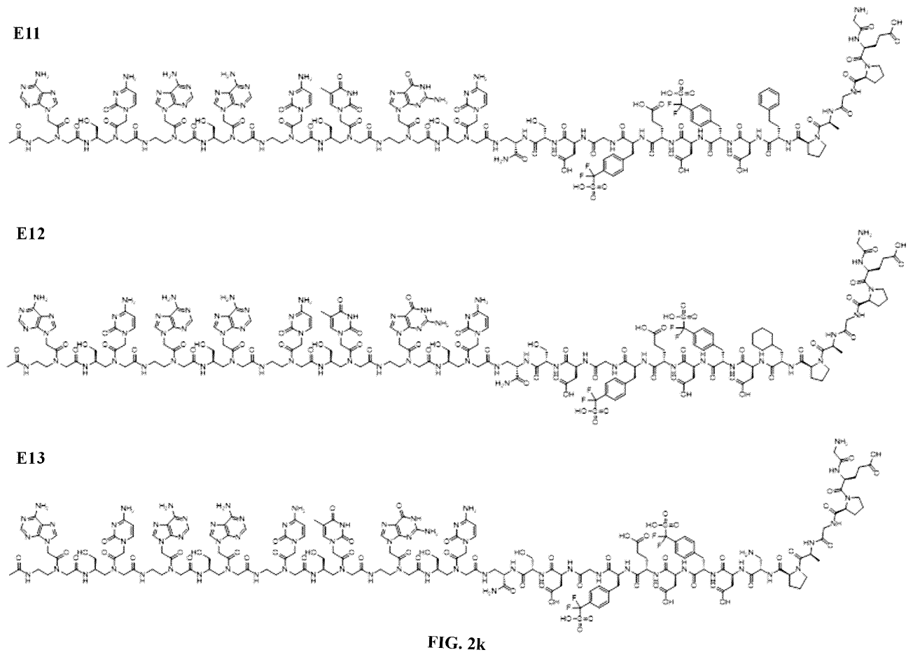

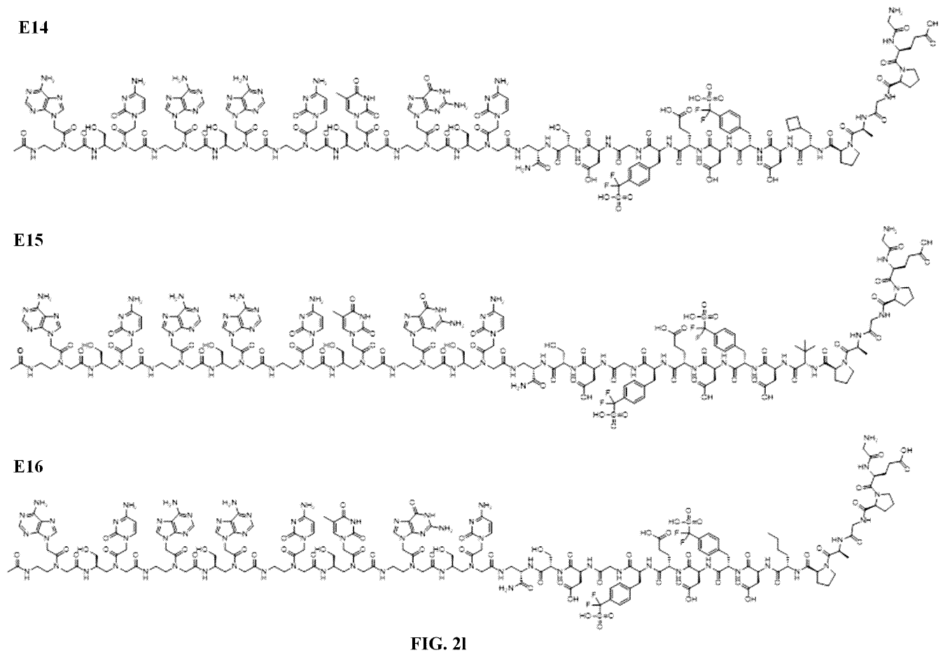

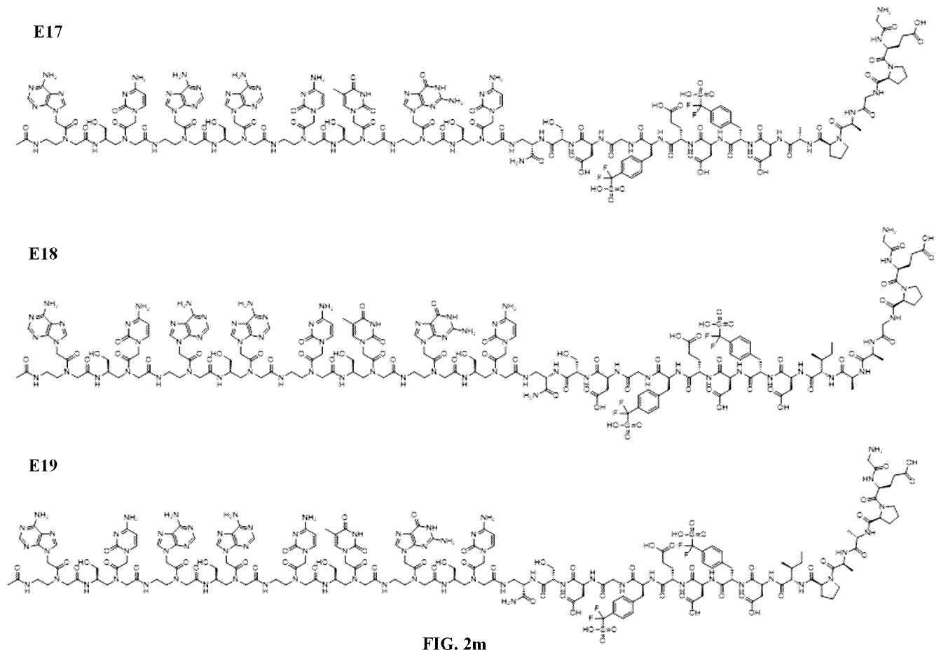

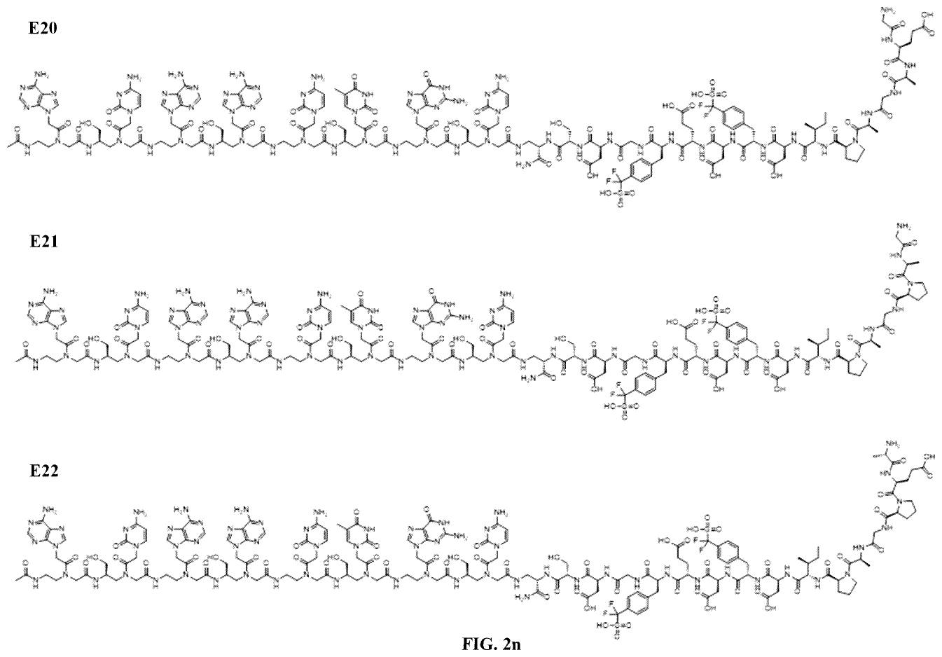

Having established promising in vivo efficacy of the supramolecular assembly inhibitor, the ability to reverse the anticoagulant activity with an antidote was investigated. Given the non-covalent nature of the supramolecular linker between the active site and exosite II binding entities, its disruption can be achieved by competing for the hybridization. PNA antidotes, AD1: DCDDCTGC and AD2: SEQ ID NO: 2 (FIGS. 2d and e).

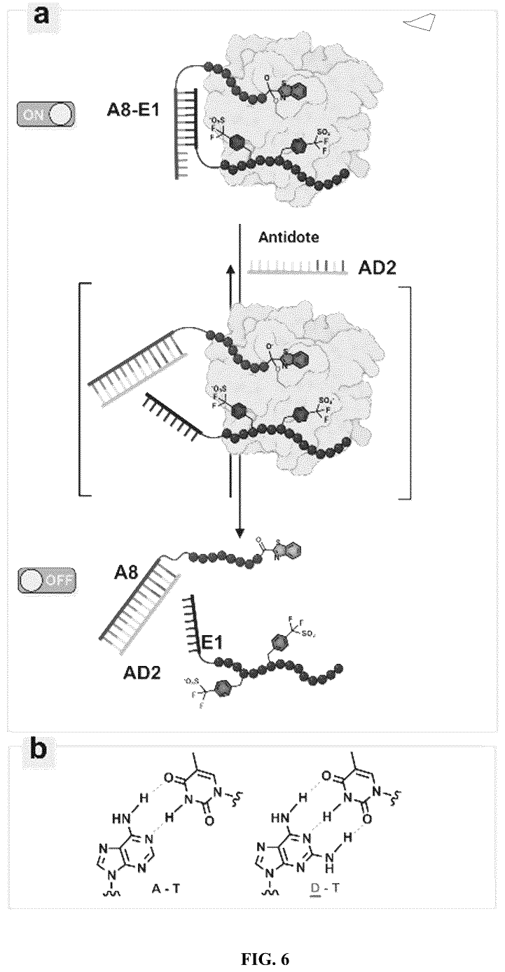

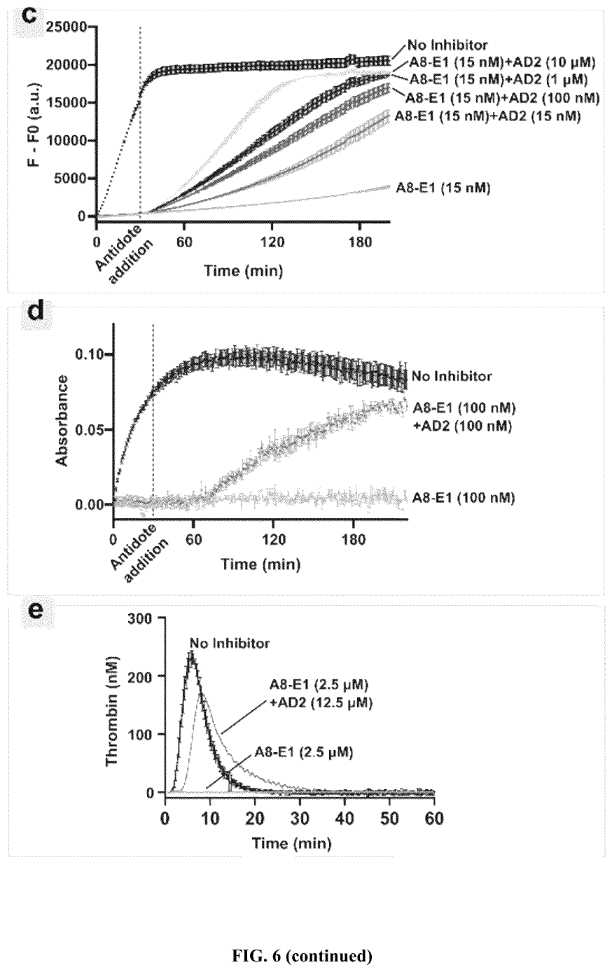

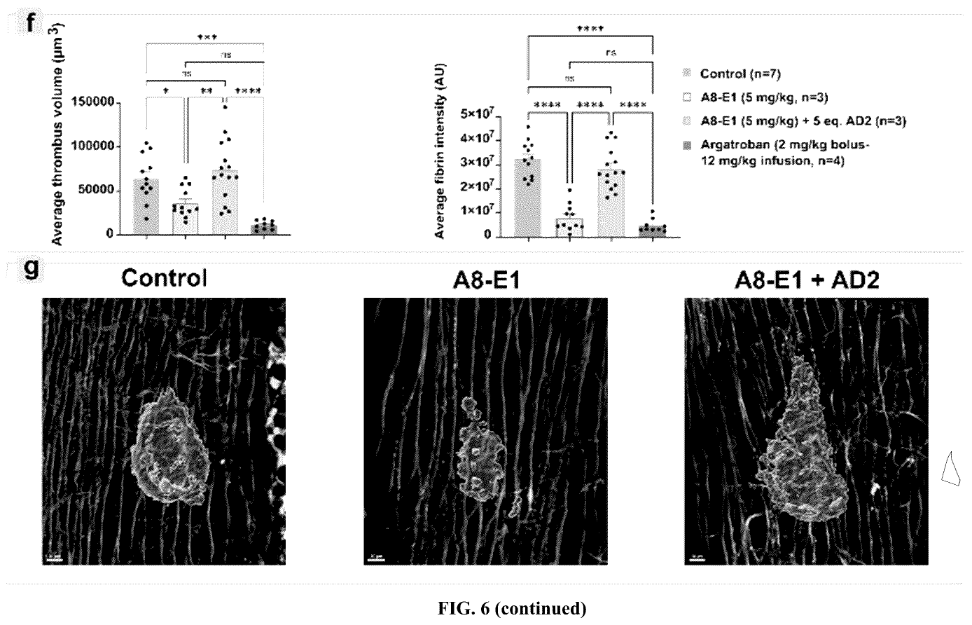

To favour the equilibrium towards the dissociation of the binary fragments, a competitor PNA was designed to incorporate diaminopurines (D) instead of adenine (A), since oligomers containing D form more stable duplexes with their complementary strand than oligomers containing A (Haaima et al., 1997, Nuc. Acids Res. 25, 4639-4643; https://doi.org:10.1093/nar/25.22.4639). While this competitor (AD1) functioned as an effective antidote by reversing inhibition, the kinetics of the antidote were deemed too slow at low concentrations (1-10 μM). A toehold sequence (Yurke et al., 2000, Nature 406, 605-608; https://doi.org:Doi 10.1038/35020524) was introduced on the supramolecular connector (A8-E1) to achieve a larger equilibrium shift in the hybridization with AD2, a 12-mer PNA (FIG. 6a-b). Following the kinetic progress of the reaction in real time with a fluorogenic substrate, the ability to switch from complete inhibition (15 nM of binary inhibitor) to ca. 40% of the uninhibited thrombin activity within 30 min using 10 μM of antidote (FIG. 6c) was observed. Using lower concentration of antidote resulted in more progressive restoration of thrombin's activity. Using just 1 equivalent of antidote was sufficient to restore ca. 20% of thrombin catalytic activity within 90 minutes. These observations were also validated in the fibrinogen clotting and CAT assays described above, with clotting restored using 5 equivalents of antidote relative to the supramolecular inhibitor (FIG. 6d-e, A8-E1+AD2). Based on these promising in vitro data, the ability of the designed antidote to reverse anticoagulation was investigated in the in vivo thrombosis model. In this experiment, first a treatment with 5 mg/kg of the supramolecular construct as described above in Example 3 was carried out (that provided effective anticoagulation in the needle injury thrombosis model) followed by administration of 5 molar equivalents of the 12-mer PNA antidote (9.4 mg/kg). Following the addition of the antidote, anticoagulation was effectively reversed as determined by the amount of fibrin deposition and thrombus volume compared to control injuries lacking the antidote (FIG. 6 f-g). These data support the potential of supramolecular inhibitors as bonafide therapeutic leads and lays the foundation for targeting a range of therapeutic targets with this approach in the future.

Altogether, those data support that the designed supramolecular anticoagulants assemblies of the invention showed potent thrombin inhibition and anticoagulation activities in vitro that could be rapidly reversed using small PNA-based antidotes. Importantly, this potent anticoagulant activity with on-demand reversibility was also demonstrated in an in vivo thrombosis model thus providing a starting point for the future use of this new therapeutic modality for bona fide anticoagulant drug candidates. PNAs are known to be metabolically stable and, unless purposefully modified, cell-impermeant (Saarbach et al., 2019, Curr Opin Chem Biol 52, 112-124; https://doi.org: 10.1016/j.cbpa.2019.06.006).

These features make PNA an ideal choice to focus the activity of supramolecular drugs to extracellular targets, limiting off target effects (Zhang et al., 2022, Nature 609, 822-828; https://doi.org:10.1038/s41586-022-05213-y) and without any intrinsic pharmacological activity for the antidote. Future improvement could make use of γ-modified PNA with a D-stereochemistry to preclude interaction with endogenous extracellular oligonucleotides (Flynn, et al., 2021, Cell 184, 3109-3124; https://doi.org:ARTN e2210.1016/j.cell.2021.04.023; Rasmussen et al., 2022, Nature 601, 422-427).

Importantly, the strategy adopted here offers a general mechanism to turn therapeutic activity on or off rapidly and is therefore not limited to applications in thrombosis. For example, the supramolecular concept would be amenable to the emerging area of immunotherapy where an antidote to a CAR-T response is highly desirable, or to immunomodulators where reversal of action is important in case of severe infection. The fact that assembly can be encoded by different sequences of low cost PNA should make it possible to multiplex programmable supramolecular drug candidates in the future.

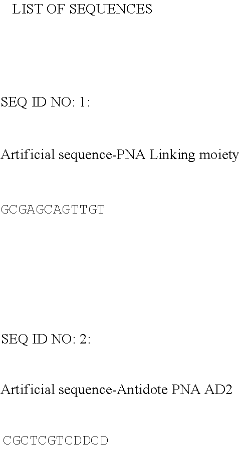

1. A method of preventing or treating a disorder in a subject in need thereof, said method comprising the steps of:
providing a first targeting agent wherein said first targeting agent comprises i) a first binding moiety that has a binding affinity to a first site of a biological target and ii) a first PNA linking moiety;
providing a second targeting agent wherein said second targeting agent comprises i) a second binding moiety that has a binding affinity to a site of the same said biological target which is different from the first site and ii) a second PNA linking moiety, wherein said first and second PNA linking moieties are at least partially complementary to each other to be able to hybridize and wherein the first binding moiety and the second binding moiety are able to interact cooperatively with the said biological target;
contacting said first targeting agent, said second targeting agent and said biological target to form a supramolecular assembly of the first and second targeting agents which are assembled through the hybridization of their complementary PNA moieties and wherein said supramolecular assembly is bound to the biological target at both the first and the second sites of said target through the first and second binding moieties.

2. The method according to claim 1, wherein the said first and said second targeting agents are contacted with the said target as a mixture or, when contacted separately, the said first and said second targeting agents are contacted with the target at similar times.

3. The method according to claim 1, wherein the method further comprises a step of administering an antidote agent to reverse the activity of the supramolecular assembly, wherein said antidote agent is a competing oligonucleotide comprising a sequence being complementary to at least a part of the first or the second PNA linking moiety.

4. The method according to claim 1, wherein the disorders are selected from a thrombotic event and a disorder treatable by immunotherapy or immunomodulation.

5. A supramolecular assembly comprising:
a first targeting agent wherein said first targeting agent comprises i) a first binding moiety that has a binding affinity to a first site of a biological target and ii) a first PNA linking moiety;
a second targeting agent wherein said second targeting agent comprises i) a second binding moiety that has a binding affinity to a site of the same said biological target which is different from the first site and ii) a second PNA linking moiety,
wherein said first and second PNA linking moieties are hybridized together through a hybridizing clamp formed by the hybridization of the parts of their PNA sequences which are complementary in presence of said biological target and wherein the first binding moiety and the second binding moiety are able to interact cooperatively with the said biological target.

6. The supramolecular assembly according to claim 5, wherein the PNA linking moieties comprise 4 to 16 nucleobases.

7. The supramolecular assembly according to claim 5, wherein the PNA linking moieties are selected from GCAGTTGT, ACAACTGC and GCGAGCAGTTGT (SEQ ID NO: 1).

8. The supramolecular assembly according to claim 5, wherein the sequence of one of the first and second PNA linking moieties comprises 1 to about 6 nucleobases (“toehold sequence”) on its free side which are not complementary to the PNA sequence of the other targeting agent.

9. The supramolecular assembly according to claim 5, wherein the biological target is thrombin and the first targeting agent is selected from A1 to A8.

10. The supramolecular assembly according to claim 5, wherein the biological target is thrombin and the second targeting agent is selected from E1 to E23.

11. A method of preventing or treating a thrombotic event in a subject in need thereof, said method comprising the steps of:
administering a combination of:
i) a first targeting agent wherein said first targeting agent comprises i) a first binding moiety that has a binding affinity to a first site of thrombin and ii) a first PNA linking moiety;
ii) a second targeting agent wherein said second targeting agent comprises i) a second binding moiety that has a binding affinity to a site of thrombin which is different from the first site and ii) a second PNA linking moiety, wherein said first and second PNA linking moieties are at least partially complementary to each other to be able to hybridize and wherein the first binding moiety and the second binding moiety are able to interact cooperatively with thrombin;
and wherein said first and second targeting agents are able to form a supramolecular assembly through the hybridization of their complementary PNA moieties and wherein said supramolecular assembly.

12. The method according to claim 11, wherein the first targeting agent is selected from A1 to A8.

13. The method according to claim 11, wherein the second targeting agent is selected from E1 to E23.

14. The method according to claim 11, wherein a supramolecular assembly formed in vivo, the supramolecular assembly comprising:
a first targeting agent wherein said first targeting agent comprises i) a first binding moiety that has a binding affinity to a first site of a biological target and ii) a first PNA linking moiety;
a second targeting agent wherein said second targeting agent comprises i) a second binding moiety that has a binding affinity to a site of the same said biological target which is different from the first site and ii) a second PNA linking moiety,
wherein said first and second PNA linking moieties are hybridized together through a hybridizing clamp formed by the hybridization of the parts of their PNA sequences which are complementary in presence of said biological target and wherein the first binding moiety and the second binding moiety are able to interact cooperatively with the said biological target.

15. The method according to claim 11, wherein an antidote agent is further administered.

16. The method according to claim 15, wherein an antidote agent selected from AD1 or AD2 is added.

17. A pharmaceutical composition comprising a first targeting agent and a second targeting agent according to claim 5, wherein said first and second PNA linking moieties are at least partially complementary to each other to be able to hybridize and wherein the first binding moiety and the second binding moiety are able to interact cooperatively with the said biological target, as well pharmaceutically acceptable salts thereof and a pharmaceutically acceptable carrier, diluent or excipient thereof.

18. The pharmaceutical composition according to claim 17, wherein said the first targeting agent is selected from A1 to A8.

19. The pharmaceutical composition according to claim 17, wherein said second targeting agent is selected from E1 to E23.

20. The pharmaceutical composition according to claim 17, further comprising a supramolecular assembly, said supramolecular assembly comprising:
a first targeting agent wherein said first targeting agent comprises i) a first binding moiety that has a binding affinity to a first site of a biological target and ii) a first PNA linking moiety;
a second targeting agent wherein said second targeting agent comprises i) a second binding moiety that has a binding affinity to a site of the same said biological target which is different from the first site and ii) a second PNA linking moiety,
wherein said first and second PNA linking moieties are hybridized together through a hybridizing clamp formed by the hybridization of the parts of their PNA sequences which are complementary in presence of said biological target and wherein the first binding moiety and the second binding moiety are able to interact cooperatively with the said biological target.
